(19) United States
(12) Patent Application Publication
(10) Pub. No.: US 2024/0117447 A1
(43) Pub. Date: Apr. 11, 2024

(54) POLYNUCLEOTIDES FOR THE AMPLIFICATION AND DETECTION OF NEISSERIA GONORRHOEAE

(71) Applicant: Talis Biomedical Corporation, Chicago, IL (US)

(72) Inventors: Andrea DEDENT, San Jose, CA (US); Shuyuan MA, San Jose, CA (US); Hedia MAAMAR, San Jose, CA (US)

(21) Appl. No.: 18/296,978

(22) Filed: Apr. 6, 2023

Related U.S. Application Data
(63) Continuation of application No. 16/349,187, filed on May 10, 2019, now abandoned.
(60) Provisional application No. 62/420,496, filed on Nov. 10, 2016.

(51) Int. Cl. C12Q 1/689; C07H 21/00; C07H 21/04; C12Q 1/6883

(52) U.S. Cl. CPC C12Q 1/689; C07H 21/00; C07H 21/04; C12Q 1/6883; C12Q 2600/118; C12Q 2600/16

(57) ABSTRACT

Disclosed herein are primers and probes related to the detection of Neisseria gonorrhoeae via nucleic acid amplification testing (NAAT), for example to amplify and determine the presence of N. gonorrhoeae nucleic acids present in test samples. Specifically the present disclosure describes primers and probes that bind to the small subunit rRNA (cytosine (967)-C(5))-methyltransferase or rsmB gene of N. gonorrhoeae for detection via loop mediated isothermal amplification (LAMP) and molecular beacon hybridization.

### CROSS REFERENCE TO RELATED APPLICATIONS

This application is a continuation of U.S. patent application Ser. No. 16/349,187, filed on May 10, 2019, now U.S. Patent Application Publication No. 2019-0284618, which is a U.S. National Phase Application of PCT/US2017/061405, filed on Nov. 13, 2017, which claims the benefit of U.S. Provisional Patent Application No. 62/420,496, filed Nov. 10, 2016, the contents of which are incorporated herein by reference in their entirety.

### GOVERNMENT LICENSE RIGHTS

This invention was made with government support under contract number HR0011-11-2-0006 awarded by the Department of Defense. The government has certain rights in the invention.

### SEQUENCE LISTING

The instant application contains a Sequence Listing which has been submitted herewith and is hereby incorporated by reference in its entirety. Said .xml copy, created on Jun. 16, 2023 is named 14672-710300 and is 132,934 bytes in size.

### FIELD OF THE INVENTION

The present invention relates to the fields of molecular biology and nucleic acid chemistry. The invention provides methods and reagents for detecting pathogens, such as Neisseria gonorrhoeae and accordingly, also relates to the fields of medical diagnostics and prognostics. In particular, the invention relates to polynucleotides and methods for amplifying and detecting Neisseria gonorrhoeae.

### BACKGROUND OF THE INVENTION

Neisseria gonorrhoeae, the etiological agent of gonorrhea, infects the urogenital tract with clinical signs of gonorrhea often overlapping with those of other sexually transmitted diseases (STDs). Infection, often asymptomatic in women, if left untreated can lead to more serious and permanent health related complications such as pelvic inflammatory disease (PID), chronic pelvic pain, tubal infertility, and life-threatening ectopic pregnancy. In men, the majority of urethral infections cause urethritis, occasionally resulting in epididymitis which can lead to infertility if not treated. Though not as common, asymptomatic infection rates among men are also significant. Among neonates, conjunctivitis can result in blindness. Among all three groups, untreated N. gonorrhoeae can disseminate leading to acute dermatitis, tenosynovitis syndrome and sepsis associated with arthritis, meningitis, or endocarditis.

N. gonorrhoeae has a global impact estimate of 106 million new cases annually. Worldwide, N. gonorrhoeae is the second most prevalent bacterial STD as well as the second most common notifiable communicable disease in the United States. The WHO estimates incidence of N. gonorrhoeae infection has been steadily rising since 1995, with an increase of 11.7% from 2005 to 2008. Compounding the clinical and increased incidence concerns is the categorization of N. gonorrhoeae as an immediate public health threat related to its antibiotic resistance profile, with 30% of strains estimated to carry resistance to one or more treatment antibiotics.

One of the main public health strategies in prevention and reduction of infectious disease is reducing person-to-person spread through screening, prompt identification and effective treatment. Imperative to this strategy are specific and sensitive diagnostics.

The performance of nucleic acid amplification tests (NAATs) in regards to sensitivity, specificity, and ease of specimen transport exceeds that of any other testing diagnostic currently available for diagnosing gonococcal infections. The CDC specifically recommends use of NAATs by clinical and disease control laboratories to detect gonorrhea with a few limited exceptions. Recommendations for the Laboratory-Based Detection of Chlamydia trachomatis and Neisseria gonorrhoeae—2014. MMWR 2014; 63(No. RR-2). Related to the sensitivity and specificity, these assays have provided for the use of less invasive specimen collection, which better facilitates infectious disease screening. Optimal recommended specimen types for NAATs include first catch urine from men and vaginal swabs from women.

At the time of preparation of this document, FDA-cleared NAATs included Abbott RealTime CT/NG (Abbott m2000 system platform), Aptima COMBO or individual CT or GC assays (Hologic Panther system platform), BD ProbeTec assays (ET CT/GC Amplified DNA assay and Q^{x} CT or GC Amplified DNA assays and BD Viper system platform), Cepheid Xpert CT/NG assay (GeneXpert IV point of care device), and Roche Diagnostics CT/NG tests (cobas 4800 system platform). The Abbott, Aptima, BD, and Roche assays all include automation for sample preparation, target amplification, and detection. While this is a benefit from a sample preparation and limited hands on time perspective, each system platform translates into a large investment in capital equipment and requires at least 3 hours to reach a sample answer. The Cepheid assay and its accompanying device is the only point of care instrument, with reduced cost, spatial fingerprint, and time to sample answer of approximately 90 minutes.

What is needed, therefore, are new assays compatible with point of care devices that offer high sensitivity, significantly reduced time to answer, reduced equipment cost, and the potential for sample in answer out utilization.

### SUMMARY

In some embodiments, provided herein is a composition comprising a set of polynucleotides selected from the group consisting of Set-1 through Set-76. In some embodiments, the composition further comprises a probe. In some embodiments, the probe comprises a label. In some embodiments, the probe is a labeled polynucleotide.

In some embodiments, the probe is a labeled polynucleotide having a sequence selected from the group consisting of SEQ ID NO: 35 (MB1), SEQ ID NO:36 (MB2), SEQ ID NO: 92 (MB7), and SEQ ID NO: 93 (MB8) and the set of polynucleotides is selected from Set-1, Set-2, Set-3, Set-4, Set-5, Set-6, Set-19, Set-20, Set-21, Set-22, Set-23, Set-24, Set-25, Set-26, Set-39, Set-40, Set-41, Set-42, Set-43, Set-56, Set-63, and Set-70.

In some embodiments, the probe is a labeled polynucleotide having a sequence selected from the group consisting of SEQ ID NO: 72 (MB6), SEQ ID NO: 94 (MB9), and SEQ ID NO: 101 (MB16), and the set of polynucleotides selected from the group consisting of Set-8, Set-28, and Set-45.

In some embodiments, the probe is a labeled polynucleotide having a sequence selected from the group consisting of SEQ ID NO: 69 (MB3), SEQ ID NO: 70 (MB4), SEQ ID NO: 71 (MB5), SEQ ID NO: 95 (MB10), SEQ ID NO: 96 (MB11), SEQ ID NO: 97 (MB12), and SEQ ID NO: 98 (MB13), and the set of polynucleotides selected from the group consisting of Set-9, Set-10, Set-11, Set-12, Set-29, Set-30, Set-31, Set-32, Set-46, Set-47, Set-48 and Set-49.

In some embodiments, t the probe is a labeled polynucleotide having a sequence selected from the group consisting of SEQ ID NO: 99 (MB14) and SEQ ID NO: 100 (MB15), and the set of polynucleotides selected from the group consisting of Set-57, Set-58, Set-59, Set-60, Set-61, Set-62, Set-64, Set-65, Set-66, Set-67, set-68, Set-69, Set-71, Set-72, Set-73, Set-74, Set-75, and Set-76.

In some embodiments, the label is a fluorophore. In some embodiments, the fluorophore is covalently attached to a terminus of the polynucleotide. In some embodiments, the probe is a molecular beacon comprising a quencher. In some embodiments, the fluorophore is FAM and the quencher is BHQ1. In other embodiments, the fluorophore is ATTO 565 or Alexa 594 and the quencher is BHQ1 or BHQ2.

Also provided herein is a molecular beacon comprising a fluorophore, a quencher and a polynucleotide, wherein the polynucleotide is selected from the group consisting of: SEQ ID NO: 35, SEQ ID NO: 36. SEQ ID NO: 69 through SEQ ID NO: 72, and SEQ ID NO: 92 through SEQ ID NO: 101. In some embodiments, the fluorophore is FAM and the quencher is BHQ1. In other embodiments, the fluorophore is ATTO 565 or Alexa 594 and the quencher is BHQ1 or BHQ2.

Also provided herein is a method of detecting Neisseria gonorrhoeae in a test sample, the method comprising: (a) extracting nucleic acid from the test sample; (b) amplifying a target sequence by reacting the nucleic acid extracted in step (a) with a reaction mixture comprising a strand displacement DNA polymerase and a sequence-specific primer set, wherein said sequence-specific primer set is selected from the group consisting of Set-1 through Set-55; and (c) detecting the presence or absence of an amplified product of step (b); wherein the presence of said amplification product is indicative of the presence of Neisseria gonorrhoeae in the test sample.

In some embodiments of the method of detecting Neisseria gonorrhoeae in a test sample, the amplification in step (b) of the target sequence is performed at between about 60° C. and 67° C. for less than 30 minutes. In some embodiments, the amplification step is performed for less than 15 minutes. In some embodiments, the amplification step is performed for less than nine minutes.

In some embodiments of the method of detecting Neisseria gonorrhoeae in a test sample, detecting the presence or absence of the amplification product comprises hybridizing the amplified product with a probe comprising a polynucleotide attached to a label.

In some embodiments of the method of detecting Neisseria gonorrhoeae in a test sample, the polynucleotide comprises a sequence selected from the group consisting of SEQ ID NO: 35 (MB1), SEQ ID NO:36 (MB2), SEQ ID NO: 92 (MB7), and SEQ ID NO: 93 (MB8), and the sequence-specific primer set is selected from Set-1, Set-2, Set-3, Set-4, Set-5, Set-6, Set-19, Set-20, Set-21, Set-22, Set-23, Set-24, Set-25, Set-26, Set-39, Set-40, Set-41, Set-42, Set-43, Set-56, Set-63, and Set-70. In some embodiments, the polynucleotide comprises a sequence selected from the group consisting of SEQ ID NO: 72 (MB6), SEQ ID NO: 94 (MB9), and SEQ ID NO: 101 (MB16), and the sequence-specific primer set is selected from S Set-8, Set-28, and Set-45. In some embodiments, the polynucleotide comprises a sequence selected from the group consisting of SEQ ID NO: 69 (MB3), SEQ ID NO: 70 (MB4), SEQ ID NO: 71 (MB5), SEQ ID NO: 95 (MB10), SEQ ID NO: 96 (MB11), SEQ ID NO: 97 (MB12), and SEQ ID NO: 98 (MB13), and the sequence-specific primer set is selected from Set-9, Set-10, Set-11, Set-12, Set-29, Set-30, Set-31, Set-32, Set-46, Set-47, Set-48 and Set-49. In some embodiments, the polynucleotide comprises a sequence selected from the group consisting of SEQ ID NO: 99 (MB14) and SEQ ID NO: 100 (MB15), and the sequence-specific primer set is selected from the group consisting of Set-57, Set-58, Set-59, Set-60, Set-61, Set-62, Set-64, Set-65, Set-66, Set-67, set-68, Set-69, Set-71, Set-72, Set-73, Set-74, Set-75, and Set-76.

In some embodiments of the method of detecting Neisseria gonorrhoeae in a test sample, the probe is a molecular beacon. In some embodiments, the reaction mixture further comprises a reverse transcriptase. In some embodiments, Neisseria gonorrhoeae is present in the test sample at a concentration of ≤100 CFU/mL. In some embodiments, Neisseria gonorrhoeae is present in the test sample at a concentration of ≤10 CFU/mL.

Also provided herein, according to some embodiments of the invention, is a kit comprising a composition comprising a set of polynucleotides selected from the group consisting of Set-1 through Set-55 and amplification reagents. In some embodiments, the amplification reagents comprise a strand displacement polymerase. In some embodiments, the kit further comprises a probe.

Also provided herein is a method of detecting Neisseria gonorrhoeae in a test sample, the method comprising: (a) extracting nucleic acid from the test sample; (b) amplifying a target sequence by reacting the nucleic acid extracted in step (a) for less than twenty minutes with a reaction mixture comprising a strand displacement DNA polymerase and a sequence-specific LAMP primer set; and (c) detecting the presence or absence of an amplified product of step (b); wherein the presence of said amplification product is indicative of the presence of Neisseria gonorrhoeae in the test sample.

In some embodiments of the method, the nucleic acid is reacted with the reaction mixture for less than fifteen minutes. In some embodiments of the method, the target sequence is located in the rRNA small subunit methyltransferase B (rsmB) gene of Neisseria gonorrhoeae. In some embodiments, the target sequence is located in the 505 ribosomal protein L6 (rplF) gene of Neisseria gonorrhoeae. In some embodiments, the target sequence is located in the 165 ribosomal subunit of Neisseria gonorrhoeae. In some embodiments of the method, the target sequence is located in the 235 ribosomal subunit of Neisseria gonorrhoeae.

In some embodiments of the method, Neisseria gonorrhoeae is present in the test sample at a concentration of ≤100 CFU/mL. In some embodiments of the method, Neisseria gonorrhoeae is present in the test sample at a concentration of ≤10 CFU/mL.

In some embodiments of the method, the test sample comprises one or more other microorganisms in addition to Neisseria gonorrhoeae, and wherein the target sequence from Neisseria gonorrhoeae is preferentially amplified over a polynucleotide sequence from the one or more other microorganisms.

In some embodiments, the invention provides a nucleic acid sequence at least 90%, at least 95%, at least 96%, at least 97%, at least 98%, at least 99%%, at least 99.1%, at least 99.2%, at least 99.3%, at least 99.4%, at least 99.5%, at least 99.6%, at least 99.7%, at least 99.8% or at least 99.9% identical to SEQ ID NOs 1-72 and methods of using those nucleic acid sequences to detect Neisseria gonorrhoeae in a test sample.

### DETAILED DESCRIPTION

Detecting low concentrations of species (down to a few molecules or microorganisms in a sample) is a challenge in medicine. The present invention relates to the selective detection of Neisseria gonorrhoeae. In particular, based on new detection strategies utilizing nucleic acid amplification, particularly RT-LAMP, and molecular beacon detection, N. gonorrhoeae infections can be diagnosed using the methods and reagents described herein. Using RNA (either ribosomal RNA (rRNA) or messenger RNA) as the target regions provides multiple copies of the target per N. gonorrhoeae genome. Accordingly, this facilitates the detection of N. gonorrhoeae in samples utilizing the approaches described herein relative to techniques that target genomic DNA, even when present in multiple copies per genome. In addition, the molecular beacon detection reagents described herein provide additional specificity, failing to bind, in most cases, to off target amplified DNA, thereby minimizing the occurrence of, e.g., false positives. This specificity is illustrated in, inter alia, Example 5 provided below. Many other features of the invention are also described herein.

As used herein, “nucleic acid” includes both DNA and RNA, including DNA and RNA containing non-standard nucleotides. A “nucleic acid” contains at least one polynucleotide (a “nucleic acid strand”). A “nucleic acid” may be single-stranded or double-stranded. The term “nucleic acid” refers to nucleotides and nucleosides which make up, for example, deoxyribonucleic acid (DNA) macromolecules and ribonucleic acid (RNA) macromolecules. Nucleic acids may be identified by the base attached to the sugar (e.g., deoxyribose or ribose).

As used herein, a “polynucleotide” refers to a polymeric chain containing two or more nucleotides, which contain deoxyribonucleotides, ribonucleotides, and/or their analog, such as those containing modified backbones (e.g. peptide nucleic acids (PNAs) or phosphorothioates) or modified bases. “Polynucleotides” includes primers, oligonucleotides, nucleic acid strands, etc. A polynucleotide may contain standard or non-standard nucleotides. Thus the term includes mRNA, tRNA, rRNA, ribozymes, DNA, cDNA, recombinant nucleic acids, branched nucleic acids, plasmids, vectors, probes, primers, etc. Typically, a polynucleotide contains a 5′ phosphate at one terminus (“5′ terminus”) and a 3′ hydroxyl group at the other terminus (“3′ terminus”) of the chain. The most 5′ nucleotide of a polynucleotide may be referred to herein as the “5′ terminal nucleotide” of the polynucleotide. The most 3′ nucleotide of a polynucleotide may be referred to herein as the “3′ terminal nucleotide” of the polynucleotide. Where nucleic acid of the invention takes the form of RNA, it may or may not have a 5′ cap.

LAMP is a nucleic acid amplification method that relies on auto-cycle strand-displacement DNA synthesis performed by Bst DNA polymerase, or other strand displacement polymerases. The amplified products are stem-loop structures with several repeated sequences of the target, and have multiple loops. The principal merit of this method is that denaturation of the DNA template is not required, and thus the LAMP reaction can be conducted under isothermal conditions (ranging from 60 to 67° C.). LAMP requires only one enzyme and four types of primers that recognize six distinct hybridization sites in the target sequence. The reaction can be accelerated by the addition of two additional primers. The method produces a large amount of amplified product, resulting in easier detection, such as detection by visual judgment of the turbidity or fluorescence of the reaction mixture.

In brief, the reaction is initiated by annealing and extension of a pair of ‘loop-forming’ primers (forward and backward inner primers, FIP and BIP, respectively), followed by annealing and extension of a pair of flanking primers (F3 and B3). Extension of these primers results in strand-displacement of the loop-forming elements, which fold up to form terminal hairpin-loop structures. Once these key structures have appeared, the amplification process becomes self-sustaining, and proceeds at constant temperature in a continuous and exponential manner (rather than a cyclic manner, like PCR) until all of the nucleotides (dATP, dTTP, dCTP & dGTP) in the reaction mixture have been incorporated into the amplified DNA. Optionally, an additional pair of primers can be included to accelerate the reaction. These primers, termed Loop primers, hybridize to non-inner primer bound terminal loops of the inner primer dumbbell shaped products.

The term “primer” as used herein refers to an oligonucleotide, which is capable of acting as a point of initiation of synthesis when placed under conditions in which synthesis of primer extension product which is complementary to a nucleic acid strand (template) is induced, i.e., in the presence of nucleotides and an agent for polymerization, such as DNA polymerase, and at a suitable temperature and pH.

LAMP allows amplification of target DNA sequences with higher sensitivity and specificity than PCR, often with reaction times of below 30 minutes, which is equivalent to the fastest real-time PCR tests. The target sequence which is amplified is typically 200-300 base-pairs (bp) in length, and the reaction relies upon recognition of between 120 bp and 160 bp of this sequence by several primers simultaneously during the amplification process. This high level of stringency makes the amplification highly specific, such that the appearance of amplified DNA in a reaction occurs only if the entire target sequence was initially present.

Applications for LAMP have been further extended to include detection of RNA molecules by addition of Reverse Transcriptase enzyme (RT). By including RNA detection, the types of targets for which LAMP can be applied are also expanded and add the ability to additionally target RNA based viruses, important regulatory non-coding RNA (sRNA, miRNA), and RNA molecules that have been associated with particular disease or physiological states. The ability to detect RNA also has the potential to increase assay sensitivity, for instance in choosing highly expressed, stable, and/or abundant messenger RNA (mRNA) or ribosomal RNA (rRNA) targets. This preliminary phase of amplification involves the reverse transcription of RNA molecules to complementary DNA (cDNA). The cDNA then serves as template for the strand displacing DNA polymerase. Use of a thermostable RT enzyme (i.e., NEB RTx) enables the reaction to be completed at a single temperature and in a one step, single mix reaction.

A “target sequence,” as used herein, means a nucleic acid sequence of Neisseria gonorrhoeae, or complement thereof, that is amplified, detected, or both amplified and detected using one or more of the polynucleotides herein provided. Additionally, while the term target sequence sometimes refers to a double stranded nucleic acid sequence, those skilled in the art will recognize that the target sequence can also be single stranded, e.g., RNA. A target sequence may be selected that is more or less specific for a particular organism. For example, the target sequence may be specific to an entire genus, to more than one genus, to a species or subspecies, serogroup, auxotype, serotype, strain, isolate or other subset of organisms.

The speed, specificity and sensitivity of the primers/probe compositions and method described herein result from several aspects. Exemplary primers for use in the compositions and methods according to the present invention include:

TABLE 1
LAMP primers

| Target | Seq ID | Sequence (5′ to 3′) |
|---|---|---|
| rsmB | SEQ ID NO: 1 | ATGCCGAAAGCTATTTGG |
| rsmB | SEQ ID NO: 2 | GCCGTCTTTCGGGTTA |
| rsmB | SEQ ID NO: 3 | CTTCTTCCAACGTAACCGCATAGT |
| | | GGCGGAAGGTATCG |
| rsmB | SEQ ID NO: 4 | GCCGGTAAACCGTCTGCCCGAAGT |
| | | CCTGTACCG |
| rsmB | SEQ ID NO: 5 | CGTCCAACGCCTTAGC |
| rsmB | SEQ ID NO: 6 | TTGCCGAAGGACTGGT |
| rsmB | SEQ ID NO: 7 | CTGGTGGCGGAAGGT |
| rsmB | SEQ ID NO: 8 | ATCCGTTCGCCGTCT |
| rsmB | SEQ ID NO: 9 | GGCACGGCTTCTTCCAAATCGCGG |
| | | CTAAGGC |
| rsmB | SEQ ID NO: 10 | TTTGCCGAAGGACTGGTGATACGC |
| | | CGCCTGC |
| rsmB | SEQ ID NO: 11 | GTAACCGCATATTCGTCCA |
| rsmB | SEQ ID NO: 12 | TCGGTACAGGACTTCGG |
| rsmB | SEQ ID NO: 13 | AAGCTATTTGGAAAAACTGGT |
| rsmB | SEQ ID NO: 14 | CTTCTTCCAACGTAACCGCATAGA |
| | | AGGTATCGCGGCT |
| rsmB | SEQ ID NO: 15 | CGTGCCGGTAAACCGTCGCCGAAG |
| | | TCCTGT |
| rsmB | SEQ ID NO: 16 | CATATTCGTCCAACGCCTTA |
| rsmB | SEQ ID NO: 17 | TGCCGAAGGACTGGT |
| rsmB | SEQ ID NO: 18 | TTGGACGAATATGCGGTT |
| rsmB | SEQ ID NO: 19 | CCGTCTGAAAGCCCAAA |
| rsmB | SEQ ID NO: 20 | AGATACGCCGCCTGCTGCGTTGGA |
| | | AGAAGCCGTG |
| rsmB | SEQ ID NO: 21 | TGGAACTGGCGGACTGCGGCGATA |
| | | TTGTCCTCCAC |
| rsmB | SEQ ID NO: 22 | CCGAAGTCCTGTACCGAC |
| rsmB | SEQ ID NO: 23 | CGTTACCGCCTTGGACA |
| rsmB | SEQ ID NO: 24 | TTGGACGAATATGCGGTTA |
| rsmB | SEQ ID NO: 25 | CGCCGTCTTTCGGGTTAAGGAGCC |
| | | GTGCCGGTA |
| rsmB | SEQ ID NO: 26 | GCATATTTTGGAACTGGCGGACGA |
| | | TATTGTCCTCCACACGC |
| rsmB | SEQ ID NO: 27 | ACGCCGAAGTCCTGT |
| rsmB | SEQ ID NO: 28 | CGTTACCGCCTTGGAC |
| rsmB | SEQ ID NO: 29 | GCAATGCCGAAAGCTATTTGG |
| rsmB | SEQ ID NO: 30 | AAATCCGTTCGCCGTCTTTC |
| rsmB | SEQ ID NO: 31 | GGCTTCTTCCAACGTAACCGCATT |
| | | GGTGGCGGAAGGTATCG |
| rsmB | SEQ ID NO: 32 | TGCCCGGTTTTGCCGAAGGTAAGG |
| | | AGATACGCCGCCTG |
| rsmB | SEQ ID NO: 33 | ATTCGTCCAACGCCTTAGCC |
| rsmB | SEQ ID NO: 34 | ACTGGTGTCGGTACAGGACTT |
| 16S | SEQ ID NO: 37 | GAGCGCAACCCTTGTCAT |
| 16S | SEQ ID NO: 38 | TCCGACTTCATGCACTCGA |
| 16S | SEQ ID NO: 39 | GAGGACTTGACGTCATCCCCACGG |
| | | GCACTCTAATGAGACTGC |
| 16S | SEQ ID NO: 40 | ATGGTCGGTACAGAGGGTAGCCAG |
| | | TGCAATCCGGACTACGAT |
| 16S | SEQ ID NO: 41 | CTTCCTCCGGCTTGTCACCG |
| 16S | SEQ ID NO: 42 | AGGCGGAGCCAATCTCACAAAAC |
| 23S | SEQ ID NO: 43 | CCGGCTAAGGTCCCAAAT |
| 23S | SEQ ID NO: 44 | TCGCACTTCTGATACCTCC |
| 23S | SEQ ID NO: 45 | TCGACCAGTGAGCTATTACGCTGA |
| | | AGTGGGAAGGCACAGA |
| 23S | SEQ ID NO: 46 | CTATAACCGAAGCTGCGGATGCAT |
| | | CAGCCTACAGAACGCTC |
| 23S | SEQ ID NO: 47 | GCTTCTAAGCCAACATCCTGG |
| 23S | SEQ ID NO: 48 | CGGTTTACCGGCATGGTAG |
| 23S | SEQ ID NO: 49 | AGAGAACTCGGGAGAAGGAA |
| 23S | SEQ ID NO: 50 | TCGCTACCTTAGGACCGTTA |
| 23S | SEQ ID NO: 51 | CAGCCACCTATTCTCTGCGACCGG |
| | | AGAAGGTATGCCCTCTAAG |
| 23S | SEQ ID NO: 52 | CGTATAGGGTGTAACGCCTGCCGG |
| | | CTTCGATCCGATGCTT |
| 23S | SEQ ID NO: 53 | GGCTTACGGAGCAAGTCCT |
| 23S | SEQ ID NO: 54 | CGGTGCCGGAAGGTTAATTG |
| 23S | SEQ ID NO: 55 | GGAGAAGGAACTCGGCAA |
| 23S | SEQ ID NO: 56 | CTTCGATCCGATGCTTGC |
| 23S | SEQ ID NO: 57 | AGCCACCTATTCTCTGCGACCGGA |
| | | GAAGGTATGCCCTCTAA |
| 23S | SEQ ID NO: 58 | GAGCACTCTTGCCAACACGAACAA |
| | | TTAACCTTCCGGCACC |
| 23S | SEQ ID NO: 59 | CTTACGGAGCAAGTCCTTAACC |
| 23S | SEQ ID NO: 60 | GTATAGGGTGTAACGCCTGC |
| 23S | SEQ ID NO: 61 | AGCACTCTGCCAACACGAACCACG |
| | | GCCTTCCAATTAAC |
| 23S | SEQ ID NO: 62 | GTATAGGGTGTCACGCCTGC |
| 23S | SEQ ID NO: 63 | AGAGAATAGGTGGCTGCGA |
| 23S | SEQ ID NO: 64 | AGACAGTGTGGCCATCGT |
| 23S | SEQ ID NO: 65 | GCAGGCGTCACACCCTATACGGTT |
| | | TATTAAAAACACAGCACTCTGCC |
| 23S | SEQ ID NO: 66 | CGGTAAACGGCGGCCGTAAATTCG |
| | | TGCGGGTCGGAAC |
| 23S | SEQ ID NO: 67 | CTATACGTCCACTTTCGTGTTG |
| 23S | SEQ ID NO: 68 | TAACGGTCCTAAGGTAGCGAA |
| rsmB | SEQ ID NO: 73 | CCGTCTGCCCGGTCGCCGAAGTCC |
| | | TGT |
| rpIF | SEQ ID NO: 74 | TTGGAACAGAGGCATTGGT |
| rpIF | SEQ ID NO: 75 | CAACTTGTTTATCCGAGCCAG |
| rpIF | SEQ ID NO: 76 | GCTGACTAATGCGCGAGCAGGCAT |
| | | TCTGATGTAGCCATTGAA |
| rpIF | SEQ ID NO: 77 | GGTTATCGTGCTCAAGCACAAGGT |
| | | CTGTTTGGCTAGGAGTTTGA |
| rpIF | SEQ ID NO: 78 | ACCAGACATTGCATTTGCTTGT |
| rpIF | SEQ ID NO: 79 | TGCCTGAAGGTGTCTCCG |
| rpIF | SEQ ID NO: 80 | GCATTCTGATGTAGCCATTG |
| rpIF | SEQ ID NO: 81 | CTCTGTTTGGCTAGGAGT |
| rpIF | SEQ ID NO: 82 | TCTTCTCAAAACCTTCTGAAACAC |
| | | CAAGCAAATGCAATGTCTGG |
| rpIF | SEQ ID NO: 83 | CGTGCTCAAGCACAAGGTAATTGA |
| | | ACGGAGACACCTTC |
| rpIF | SEQ ID NO: 84 | ACCATATTGCTGACTAATGCG |
| rpIF | SEQ ID NO: 85 | ATCCGATCGTATATGAAATGCC |
| rpIF | SEQ ID NO: 86 | GCAGTAAACAAGCAAATGC |
| rpIF | SEQ ID NO: 87 | TCTTCTCAAAACCTTCTGAAACAC |
| | | CAATGTCTGGTACTGCTCG |
| rpIF | SEQ ID NO: 88 | CTTCTCAAAACCTTCTGAAACACC |
| | | TAATGTCTGGTACTGCTCG |
| rpIF | SEQ ID NO: 89 | TCTTCTCAAAACCTTCTGAAACAC |
| | | CGCAGTAAACAAGCAAATGC |
| rpIF | SEQ ID NO: 90 | CCATTGAATTTAATGATGGCAAAT |
| | | TGA |
| rpIF | SEQ ID NO: 91 | CGTGGGTTATCGTGCTCAAGCTTG |
| | | AACGGAGACACCTTC |

Detection of the LAMP amplified products can be achieved via a variety of methods. In a preferred embodiment, detection of product is conducted by adding a fluorescently-labeled probe to the primer mix. The term used herein “probe” refers to a single-stranded nucleic acid molecule comprising a portion or portions that are complementary, or substantially complementary, to a target sequence. In certain implementations, the fluorescently-labeled probe is a molecular beacon.

As used herein, “molecular beacon” refers to a single stranded hairpin-shaped oligonucleotide probe designed to report the presence of specific nucleic acids in a solution. A molecular beacon consists of four components; a stem, hairpin loop, end labelled fluorophore and opposite end-labelled quencher (Tyagi et al., (1998) Nature Biotechnology 16:49-53). When the hairpin-like beacon is not bound to a target, the fluorophore and quencher lie close together and fluorescence is suppressed. In the presence of a complementary target nucleotide sequence, the stem of the beacon opens to hybridize to the target. This separates the fluorophore and quencher, allowing the fluorophore to fluoresce. Alternatively, molecular beacons also include fluorophores that emit in the proximity of an end-labelled donor. “Wavelength-shifting Molecular Beacons” incorporate an additional harvester fluorophore enabling the fluorophore to emit more strongly. Current reviews of molecular beacons include Wang et al., 2009, Angew Chem Int Ed Engl, 48(5):856-870; Cissell et al., 2009, Anal Bioanal Chem 393(1):125-35; Li et al., 2008, Biochem Biophys Res Comm 373(4):457-61; and Cady, 2009, Methods Mol Biol 554:367-79.

The term “label” as used herein means a molecule or moiety having a property or characteristic which is capable of detection and, optionally, of quantitation. A label can be directly detectable, as with, for example (and without limitation), radioisotopes, fluorophores, chemiluminophores, enzymes, colloidal particles, fluorescent microparticles and the like; or a label may be indirectly detectable, as with, for example, specific binding members. It will be understood that directly detectable labels may require additional components such as, for example, substrates, triggering reagents, quenching moieties, light, and the like to enable detection and/or quantitation of the label. When indirectly detectable labels are used, they are typically used in combination with a “conjugate”. A conjugate is typically a specific binding member that has been attached or coupled to a directly detectable label. Coupling chemistries for synthesizing a conjugate are well known in the art and can include, for example, any chemical means and/or physical means that does not destroy the specific binding property of the specific binding member or the detectable property of the label. As used herein, “specific binding member” means a member of a binding pair, i.e., two different molecules where one of the molecules through, for example, chemical or physical means specifically binds to the other molecule. In addition to antigen and antibody specific binding pairs, other specific binding pairs include, but are not intended to be limited to, avidin and biotin; haptens and antibodies specific for haptens; complementary nucleotide sequences; enzyme cofactors or substrates and enzymes; and the like.

The molecular beacon can be composed of nucleic acid only such as DNA or RNA, or it can be composed of a peptide nucleic acid (PNA) conjugate. The fluorophore can be any fluorescent organic dye or a single quantum dot. The quenching moiety desirably quenches the luminescence of the fluorophore.

Any suitable quenching moiety that quenches the luminescence of the fluorophore can be used. A fluorophore can be any fluorescent marker/dye known in the art. Examples of suitable fluorescent markers include, but are not limited to, Fam, Hex, Tet, Joe, Rox, Tamra, Max, Edans, Cy dyes such as Cy5, Fluorescein, Coumarin, Eosine, Rhodamine, Bodipy, Alexa, Cascade Blue, Yakima Yellow, Lucifer Yellow, Texas Red, and the family of ATTO dyes. A quencher can be any quencher known in the art. Examples of quenchers include, but are not limited to, Dabcyl, Dark Quencher, Eclipse Dark Quencher, ElleQuencher, Tamra, BHQ and QSY (all of them are Trade-Marks). The skilled person would know which combinations of dye/quencher are suitable when designing a probe. In an exemplary embodiment, fluorescein (FAM) is used in conjunction with Blackhole Quencher™ (BHQ™)(Novato, Calif.). Binding of the molecular beacon to amplified product can then be directly, visually assessed. Alternatively, the fluorescence level can be measured by spectroscopy in order to improve sensitivity.

A variety of commercial suppliers produce standard and custom molecular beacons, including Abingdon Health (UK; www.abingdonhealth.com), Attostar (US, MN; www.attostar.com), Biolegio (NLD; www.biolegio.com), Biomers.net (DEU; www.biomers.net), Biosearch Technologies (US, CA; www.biosearchtech.com), Eurogentec (BEL; www.eurogentec.com), Gene Link (US, NY; www.genelink.com) Integrated DNA Technologies (US, IA; www.idtdna.com), Isogen Life Science (NLD; www.isogen-lifescience.com), Midland Certified Reagent (US, TX; www.oligos.com), Eurofins (DEU; www.eurofinsgenomics.eu), Sigma-Aldrich (US, TX; www.sigmaaldrich.com), Thermo Scientific (US, MA; www.thermoscientific.com), TIB MOLBIOL (DEU; www.tib-molbiol.de), TriLink Bio Technologies (US, CA; www.trilinkbiotech.com). A variety of kits, which utilize molecular beacons are also commercially available, such as the Sentinel™ Molecular Beacon Allelic Discrimination Kits from Stratagene (La Jolla, Calif.) and various kits from Eurogentec SA (Belgium, eurogentec.com) and Isogen Bioscience BV (The Netherlands, isogen.com).

The oligonucleotide probes and primers of the invention are optionally prepared using essentially any technique known in the art. In certain embodiments, for example, the oligonucleotide probes and primers described herein are synthesized chemically using essentially any nucleic acid synthesis method, including, e.g., according to the solid phase phosphoramidite triester method described by Beaucage and Caruthers (1981), Tetrahedron Setts. 22(20):1859-1862, which is incorporated by reference, or another synthesis technique known in the art, e.g., using an automated synthesizer, as described in Needham-VanDevanter et al. (1984) Nucleic Acids Res. 12:6159-6168, which is incorporated by reference. A wide variety of equipment is commercially available for automated oligonucleotide synthesis. Multi-nucleotide synthesis approaches (e.g., tri-nucleotide synthesis, etc.) are also optionally utilized. Moreover, the primer nucleic acids described herein optionally include various modifications. To further illustrate, primers are also optionally modified to improve the specificity of amplification reactions as described in, e.g., U.S. Pat. No. 6,001,611, issued Dec. 14, 1999, which is incorporated by reference. Primers and probes can also be synthesized with various other modifications as described herein or as otherwise known in the art.

In addition, essentially any nucleic acid (and virtually any labeled nucleic acid, whether standard or non-standard) can be custom or standard ordered from any of a variety of commercial sources, such as Integrated DNA Technologies, the Midland Certified Reagent Company, Eurofins, Biosearch Technologies, Sigma Aldrich and many others.

Test samples are generally derived or isolated from subjects, typically mammalian subjects, more typically human subjects, suspected of having a N. gonorrhoeae infection. Exemplary samples or specimens include blood, plasma, serum, urine, synovial fluid, seminal fluid, seminal plasma, prostatic fluid, vaginal fluid, cervical fluid, uterine fluid, cervical scrapings, amniotic fluid, anal scrapings, mucus, sputum, tissue, and the like. Essentially any technique for acquiring these samples is optionally utilized including, e.g., scraping, venipuncture, swabbing, biopsy, or other techniques known in the art.

The term “test sample” as used herein, means a sample taken from an organism or biological fluid that is suspected of containing or potentially contains a target sequence. The test sample can be taken from any biological source, such as for example, tissue, blood, saliva, sputa, mucus, sweat, urine, urethral swabs, cervical swabs, vaginal swabs, urogenital or anal swabs, conjunctival swabs, ocular lens fluid, cerebral spinal fluid, milk, ascites fluid, synovial fluid, peritoneal fluid, amniotic fluid, fermentation broths, cell cultures, chemical reaction mixtures and the like. The test sample can be used (i) directly as obtained from the source or (ii) following a pre-treatment to modify the character of the sample. Thus, the test sample can be pre-treated prior to use by, for example, preparing plasma or serum from blood, disrupting cells or viral particles, preparing liquids from solid materials, diluting viscous fluids, filtering liquids, distilling liquids, concentrating liquids, inactivating interfering components, adding reagents, purifying nucleic acids, and the like.

Advantageously, the invention enables reliable rapid detection of Neisseria gonorrhoeae in a clinical sample, such as a urine sample.

To further illustrate, prior to analyzing the target nucleic acids described herein, those nucleic acids may be purified or isolated from samples that typically include complex mixtures of different components. Cells in collected samples are typically lysed to release the cell contents. For example, N. gonorrhoeae and other cells in the particular sample can be lysed by contacting them with various enzymes, chemicals, and/or lysed by other approaches known in the art, which degrade, e.g., bacterial cell walls. In some embodiments, nucleic acids are analyzed directly in the cell lysate. In other embodiments, nucleic acids are further purified or extracted from cell lysates prior to detection. Essentially any nucleic acid extraction methods can be used to purify nucleic acids in the samples utilized in the methods of the present invention. Exemplary techniques that can be used to purifying nucleic acids include, e.g., affinity chromatography, hybridization to probes immobilized on solid supports, liquid-liquid extraction (e.g., phenol-chloroform extraction, etc.), precipitation (e.g., using ethanol, etc.), extraction with filter paper, extraction with micelle-forming reagents (e.g., cetyl-trimethyl-ammonium-bromide, etc.), binding to immobilized intercalating dyes (e.g., ethidium bromide, acridine, etc.), adsorption to silica gel or diatomic earths, adsorption to magnetic glass particles or organo silane particles under chaotropic conditions, and/or the like. Sample processing is also described in, e.g., U.S. Pat. Nos. 5,155,018, 6,383,393, and 5,234,809, which are each incorporated by reference.

A test sample may optionally have been treated and/or purified according to any technique known by the skilled person, to improve the amplification efficiency and/or qualitative accuracy and/or quantitative accuracy. The sample may thus exclusively, or essentially, consist of nucleic acid(s), whether obtained by purification, isolation, or by chemical synthesis. Means are available to the skilled person, who would like to isolate or purify nucleic acids, such as DNA, from a test sample, for example to isolate or purify DNA from cervical scrapes (e.g., QIAamp-DNA Mini-Kit; Qiagen, Hilden, Germany).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Target Selection, Sequence Analysis and Assay Design

Sequences for Neisseria gonorrhoeae and closely related species including Neisseria meningitidis, Neisseria lactamica, and Neisseria sicca were obtained from the National Center for Biotechnology Information (NCBI) or Pathosystems Resource Integration Center (PATRIC) databases. Sequences were aligned using Clustal Omega (Sievers, et al. (2011). Molecular Systems Biology 7:539) or MAFFT (Katoh, Standley 2013. Molecular Biology and Evolution 30:772-780) and regions unique to N. gonorrhoeae were selected for primer and molecular beacon probe design.

Primer/probe based detection assays were designed to utilize isothermal loop mediated amplification (LAMP) targeting RNA through the addition of a Reverse transcriptase (RT-LAMP) to the reaction. A molecular beacon probe with 5′ fluorophore/3′ quencher modifications (6-Carboxyfluorescein and Black Hole Quencher 1 in most instances or Atto 565N and Black Hole Quencher 2 where indicated) was included to provide target-specific fluorescent detection. N. gonorrhoeae RT-LAMP primer sets (Table 1 and Table 2) were designed using a combination of software programs including PremierBiosoft's LAMP Designer, Beacon Designer, an in-house command line based script and manual designs. Resulting assay amplicons and molecular beacons were additionally Blasted against the NCBI nucleotide database, including the human transcriptome, and against individual non-gonorrhoeae species within the genus Neisseria to further predict assay specificity.

The inventive primer sets are summarized in Table 2, which include, at a minimum, a forward inner primer (FIP) and backward inner primer (BIP). Additionally, the primer sets typically also include at least two additional primers selected from the forward outer primer (F3), backward outer primer (B3), forward loop primer (LF) and backward loop primer (LB).

TABLE 2
LAMP Primer Sets

| Set | F3 | B3 | FIP | BIP | LF | LB |
|---|---|---|---|---|---|---|
| Set-1 | SEQ ID NO: 1 | SEQ ID NO: 2 | SEQ ID NO: 3 | SEQ ID NO: 4 | SEQ ID NO: 5 | SEQ ID NO: 6 |
| Set-2 | SEQ ID NO: 7 | SEQ ID NO: 8 | SEQ ID NO: 9 | SEQ ID NO: 10 | SEQ ID NO: 11 | SEQ ID NO: 12 |
| Set-3 | SEQ ID NO: 13 | SEQ ID NO: 2 | SEQ ID NO: 14 | SEQ ID NO: 15 | SEQ ID NO: 16 | SEQ ID NO: 17 |
| Set-4 | SEQ ID NO: 18 | SEQ ID NO: 19 | SEQ ID NO: 20 | SEQ ID NO: 21 | SEQ ID NO: 22 | SEQ ID NO: 23 |
| Set-5 | SEQ ID NO: 24 | SEQ ID NO: 19 | SEQ ID NO: 25 | SEQ ID NO: 26 | SEQ ID NO: 27 | SEQ ID NO: 28 |
| Set-6 | SEQ ID NO: 29 | SEQ ID NO: 30 | SEQ ID NO: 31 | SEQ ID NO: 32 | SEQ ID NO: 33 | SEQ ID NO: 34 |
| Set-7 | SEQ ID NO: 37 | SEQ ID NO: 38 | SEQ ID NO: 39 | SEQ ID NO: 40 | SEQ ID NO: 41 | SEQ ID NO: 42 |
| Set-8 | SEQ ID NO: 43 | SEQ ID NO: 44 | SEQ ID NO: 45 | SEQ ID NO: 46 | SEQ ID NO: 47 | SEQ ID NO: 48 |
| Set-9 | SEQ ID NO: 49 | SEQ ID NO: 50 | SEQ ID NO: 51 | SEQ ID NO: 52 | SEQ ID NO: 53 | SEQ ID NO: 54 |
| Set-10 | SEQ ID NO: 55 | SEQ ID NO: 56 | SEQ ID NO: 57 | SEQ ID NO: 58 | SEQ ID NO: 59 | SEQ ID NO: 60 |
| Set-11 | SEQ ID NO: 55 | SEQ ID NO: 56 | SEQ ID NO: 57 | SEQ ID NO: 61 | SEQ ID NO: 59 | SEQ ID NO: 62 |
| Set-12 | SEQ ID NO: 63 | SEQ ID NO: 64 | SEQ ID NO: 65 | SEQ ID NO: 66 | SEQ ID NO: 67 | SEQ ID NO: 68 |
| Set-13 | SEQ ID NO: 13 | SEQ ID NO: 2 | SEQ ID NO: 14 | SEQ ID NO: 15 | SEQ ID NO: 16 | SEQ ID NO: 17 |
| Set-14 | SEQ ID NO: 18 | SEQ ID NO: 19 | SEQ ID NO: 20 | SEQ ID NO: 21 | SEQ ID NO: 22 | SEQ ID NO: 23 |
| Set-15 | SEQ ID NO: 24 | SEQ ID NO: 19 | SEQ ID NO: 25 | SEQ ID NO: 26 | SEQ ID NO: 27 | SEQ ID NO: 28 |
| Set-16 | SEQ ID NO: 29 | SEQ ID NO: 30 | SEQ ID NO: 31 | SEQ ID NO: 32 | SEQ ID NO: 33 | SEQ ID NO: 34 |
| Set-17 | SEQ ID NO: 43 | SEQ ID NO: 44 | SEQ ID NO: 45 | SEQ ID NO: 46 | SEQ ID NO: 47 | SEQ ID NO: 48 |
| Set-18 | SEQ ID NO: 49 | SEQ ID NO: 50 | SEQ ID NO: 51 | SEQ ID NO: 52 | SEQ ID NO: 53 | SEQ ID NO: 54 |
| Set-19 | — | — | SEQ ID NO: 3 | SEQ ID NO: 4 | SEQ ID NO: 5 | SEQ ID NO: 6 |
| Set-20 | — | — | SEQ ID NO: 3 | SEQ ID NO: 4 | — | — |
| Set-21 | SEQ ID NO: 1 | SEQ ID NO: 2 | SEQ ID NO: 3 | SEQ ID NO: 4 | — | — |
| Set-22 | SEQ ID NO: 7 | SEQ ID NO: 8 | SEQ ID NO: 9 | SEQ ID NO: 10 | — | — |
| Set-23 | SEQ ID NO: 13 | SEQ ID NO: 2 | SEQ ID NO: 14 | SEQ ID NO: 15 | — | — |
| Set-24 | SEQ ID NO: 18 | SEQ ID NO: 19 | SEQ ID NO: 20 | SEQ ID NO: 21 | — | — |
| Set-25 | SEQ ID NO: 24 | SEQ ID NO: 19 | SEQ ID NO: 25 | SEQ ID NO: 26 | — | — |
| Set-26 | SEQ ID NO: 29 | SEQ ID NO: 30 | SEQ ID NO: 31 | SEQ ID NO: 32 | — | — |
| Set-27 | SEQ ID NO: 37 | SEQ ID NO: 38 | SEQ ID NO: 39 | SEQ ID NO: 40 | — | — |
| Set-28 | SEQ ID NO: 43 | SEQ ID NO: 44 | SEQ ID NO: 45 | SEQ ID NO: 46 | — | — |
| Set-29 | SEQ ID NO: 49 | SEQ ID NO: 50 | SEQ ID NO: 51 | SEQ ID NO: 52 | — | — |
| Set-30 | SEQ ID NO: 55 | SEQ ID NO: 56 | SEQ ID NO: 57 | SEQ ID NO: 58 | — | — |
| Set-31 | SEQ ID NO: 55 | SEQ ID NO: 56 | SEQ ID NO: 57 | SEQ ID NO: 61 | — | — |
| Set-32 | SEQ ID NO: 63 | SEQ ID NO: 64 | SEQ ID NO: 65 | SEQ ID NO: 66 | — | — |
| Set-33 | SEQ ID NO: 13 | SEQ ID NO: 2 | SEQ ID NO: 14 | SEQ ID NO: 15 | — | — |
| Set-34 | SEQ ID NO: 18 | SEQ ID NO: 19 | SEQ ID NO: 20 | SEQ ID NO: 21 | — | — |
| Set-35 | SEQ ID NO: 24 | SEQ ID NO: 19 | SEQ ID NO: 25 | SEQ ID NO: 26 | — | — |
| Set-36 | SEQ ID NO: 29 | SEQ ID NO: 30 | SEQ ID NO: 31 | SEQ ID NO: 32 | — | — |
| Set-37 | SEQ ID NO: 43 | SEQ ID NO: 44 | SEQ ID NO: 45 | SEQ ID NO: 46 | — | — |
| Set-38 | SEQ ID NO: 49 | SEQ ID NO: 50 | SEQ ID NO: 51 | SEQ ID NO: 52 | — | — |
| Set-39 | — | — | SEQ ID NO: 9 | SEQ ID NO: 10 | SEQ ID NO: 11 | SEQ ID NO: 12 |
| Set-40 | — | — | SEQ ID NO: 14 | SEQ ID NO: 15 | SEQ ID NO: 16 | SEQ ID NO: 17 |
| Set-41 | — | — | SEQ ID NO: 20 | SEQ ID NO: 21 | SEQ ID NO: 22 | SEQ ID NO: 23 |
| Set-42 | — | — | SEQ ID NO: 25 | SEQ ID NO: 26 | SEQ ID NO: 27 | SEQ ID NO: 28 |
| Set-43 | — | — | SEQ ID NO: 31 | SEQ ID NO: 32 | SEQ ID NO: 33 | SEQ ID NO: 34 |
| Set-44 | — | — | SEQ ID NO: 39 | SEQ ID NO: 40 | SEQ ID NO: 41 | SEQ ID NO: 42 |
| Set-45 | — | — | SEQ ID NO: 45 | SEQ ID NO: 46 | SEQ ID NO: 47 | SEQ ID NO: 48 |
| Set-46 | — | — | SEQ ID NO: 51 | SEQ ID NO: 52 | SEQ ID NO: 53 | SEQ ID NO: 54 |
| Set-47 | — | — | SEQ ID NO: 57 | SEQ ID NO: 58 | SEQ ID NO: 59 | SEQ ID NO: 60 |
| Set-48 | — | — | SEQ ID NO: 57 | SEQ ID NO: 61 | SEQ ID NO: 59 | SEQ ID NO: 62 |
| Set-49 | — | — | SEQ ID NO: 65 | SEQ ID NO: 66 | SEQ ID NO: 67 | SEQ ID NO: 68 |
| Set-50 | — | — | SEQ ID NO: 14 | SEQ ID NO: 15 | SEQ ID NO: 16 | SEQ ID NO: 17 |
| Set-51 | — | — | SEQ ID NO: 20 | SEQ ID NO: 21 | SEQ ID NO: 22 | SEQ ID NO: 23 |
| Set-52 | — | — | SEQ ID NO: 25 | SEQ ID NO: 26 | SEQ ID NO: 27 | SEQ ID NO: 28 |
| Set-53 | — | — | SEQ ID NO: 31 | SEQ ID NO: 32 | SEQ ID NO: 33 | SEQ ID NO: 34 |
| Set-54 | — | — | SEQ ID NO: 45 | SEQ ID NO: 46 | SEQ ID NO: 47 | SEQ ID NO: 48 |
| Set-55 | — | — | SEQ ID NO: 51 | SEQ ID NO: 52 | SEQ ID NO: 53 | SEQ ID NO: 54 |
| Set-56 | SEQ ID NO: 13 | SEQ ID NO: 2 | SEQ ID NO: 14 | SEQ ID NO: 73 | SEQ ID NO: 16 | SEQ ID NO: 17 |
| Set-57 | SEQ ID NO: 74 | SEQ ID NO: 75 | SEQ ID NO: 76 | SEQ ID NO: 77 | SEQ ID NO: 78 | SEQ ID NO: 79 |
| Set-58 | SEQ ID NO: 80 | SEQ ID NO: 81 | SEQ ID NO: 82 | SEQ ID NO: 83 | SEQ ID NO: 84 | SEQ ID NO: 85 |
| Set-59 | SEQ ID NO: 86 | SEQ ID NO: 81 | SEQ ID NO: 87 | SEQ ID NO: 83 | SEQ ID NO: 84 | SEQ ID NO: 85 |
| Set-60 | SEQ ID NO: 86 | SEQ ID NO: 81 | SEQ ID NO: 88 | SEQ ID NO: 83 | SEQ ID NO: 84 | SEQ ID NO: 85 |
| Set-61 | SEQ ID NO: 80 | SEQ ID NO: 81 | SEQ ID NO: 89 | SEQ ID NO: 83 | SEQ ID NO: 84 | SEQ ID NO: 85 |
| Set-62 | SEQ ID NO: 90 | SEQ ID NO: 81 | SEQ ID NO: 88 | SEQ ID NO: 91 | SEQ ID NO: 84 | SEQ ID NO: 85 |
| Set-63 | SEQ ID NO: 13 | SEQ ID NO: 2 | SEQ ID NO: 14 | SEQ ID NO: 73 | — | — |
| Set-64 | SEQ ID NO: 74 | SEQ ID NO: 75 | SEQ ID NO: 76 | SEQ ID NO: 77 | — | — |
| Set-65 | SEQ ID NO: 80 | SEQ ID NO: 81 | SEQ ID NO: 82 | SEQ ID NO: 83 | — | — |
| Set-66 | SEQ ID NO: 86 | SEQ ID NO: 81 | SEQ ID NO: 87 | SEQ ID NO: 83 | — | — |
| Set-67 | SEQ ID NO: 86 | SEQ ID NO: 81 | SEQ ID NO: 88 | SEQ ID NO: 83 | — | — |
| Set-68 | SEQ ID NO: 80 | SEQ ID NO: 81 | SEQ ID NO: 89 | SEQ ID NO: 83 | — | — |
| Set-69 | SEQ ID NO: 90 | SEQ ID NO: 81 | SEQ ID NO: 88 | SEQ ID NO: 91 | — | — |
| Set-70 | — | — | SEQ ID NO: 14 | SEQ ID NO: 73 | SEQ ID NO: 16 | SEQ ID NO: 17 |
| Set-71 | — | — | SEQ ID NO: 76 | SEQ ID NO: 77 | SEQ ID NO: 78 | SEQ ID NO: 79 |
| Set-72 | — | — | SEQ ID NO: 82 | SEQ ID NO: 83 | SEQ ID NO: 84 | SEQ ID NO: 85 |
| Set-73 | — | — | SEQ ID NO: 87 | SEQ ID NO: 83 | SEQ ID NO: 84 | SEQ ID NO: 85 |
| Set-74 | — | — | SEQ ID NO: 88 | SEQ ID NO: 83 | SEQ ID NO: 84 | SEQ ID NO: 85 |
| Set-75 | — | — | SEQ ID NO: 89 | SEQ ID NO: 83 | SEQ ID NO: 84 | SEQ ID NO: 85 |
| Set-76 | — | — | SEQ ID NO: 88 | SEQ ID NO: 91 | SEQ ID NO: 84 | SEQ ID NO: 85 |

Typically, 3 to 5 μL of extracted nucleic acid material prepared as described (vide supra), titred genomic DNA (gDNA, Zeptometrix, CN #0801482DNA-10UG) where indicated, or negative controls (NU=negative urine or nuclease free water; NTC=no template control) served as template for RTLAMP reactions. 25 μl total volume reactions were prepared on ice as master mixes containing 1×NEB Isothermal amplification buffer supplemented with 5 mM KCl, 4.8 mM MgSO₄ and 1.6 mM each dCTP, dGTP, dATP and dTTP. NEB Bst2 polymerase (NEB CN #M0537L) and RTx Warmstart reverse transcriptase (NEB CN #M03805) were used at 8 and 7.5 units/reaction, respectively. Primers (2 μM inner primers, 0.2 μM outer primers, and 0.8 μM Loop primers) were added to individual reactions or directly to master mixes as required per experimental design. Molecular beacons (0.2 μM) or 200 nM Yo-Pro-1 dye also was added to the master mix, as indicated in the examples below. Where indicated, amplification reactions were prepared in which the standard 6 primer mix was replaced with a 2 (2 μM inner primers) or 4 (2 μM inner primers and 0.8 μM Loop primers) primer mix. Master mixes were distributed to individual sample templates, vortexed and centrifuged briefly and each reaction loaded into individual wells of a 96 well plate (Roche CN #4729692001). Reactions were carried out at 63° C. and fluorescence monitored on either a Roche LightCycler 96 Real-Time PCR instrument or a BioRad CFX96 real time cycler. Target amplification was monitored via molecular beacon probe binding to target resulting in release of molecular beacon fluorescence intramolecular quenching.

### Example 2: LAMP with Dye Detection

A negative urine matrix was spiked with titred N. gonorrhoeae (serially diluted in PBS, Zeptometrix CN #0801482) at 10 CFU/ml. Nucleic acids were extracted from the spiked sample or from negative urine using standard extraction methods and the sample was amplified using LAMP primer sets 1-12, as described in Table 2.

YoPro™ dye (Life Technologies; green fluorescent carbocyanine nucleic acid stain) was used for the detection of the amplified product. The master mix was prepared as described in Example 1. Results are summarized in Table 3, in which the Time to Positive (Tp) was calculated by the instrument. Results are classified by the time to positive (Tp) from reaction initiation as follows: “A” indicates a Tp of less than or equal to 8 minutes, “B” indicates a Tp of between 8 minutes and 12 minutes (inclusive), “C” indicates a Tp of between 12 minutes and 25 minutes (inclusive), and “D” indicates a Tp of greater than 25 minutes or no amplification detected (No Call).

TABLE 3
Time to Positive (Dye Detection)

| Set | 10 CFU/mL | Neg. Urine | NTC |
|---|---|---|---|
| Set-1 | A | D | D |
| Set-2 | A | D | C |
| Set-3 | D | D | D |
| Set-4 | C | D | C |
| Set-5 | C | C | B |
| Set-6 | A | D | D |
| Set-7 | D | D | D |
| Set-8 | A | C | C |
| Set-9 | A | D | B |
| Set-10 | A | D | D |
| Set-11 | B | D | D |
| Set-12 | B | C | C |

### Example 3: Molecular Beacon Detection

A subset of the primer sets described in Example 2 were additionally tested for specificity by comparing reactions with 10⁹ copies of N. gonorrhoeae gDNA template (NG) to reactions with 10⁹ copies of gDNA from closely related Neisseria species, Neisseria meningitides (NM), Neisseria lactamica (NL), and Neisseria sicca (NS). When the amplification reactions were performed as described in Example 1, each of the primer sets tested had significant cross-reactivity against additional Neisseria species (Table 4). As expected, due to the high concentration of template, the LAMP reactions occur very quickly. Results are classified by the time to positive (Tp) from reaction initiation as follows: “A” indicates a Tp of less than or equal to 5 minutes, “B” indicates a Tp of between 5 minutes and 8 minutes (inclusive), “C” indicates a Tp of between 8 minutes and 15 minutes (inclusive), and “D” indicates a Tp of greater than 26 minutes or no amplification detected.

TABLE 4
Cross-Reactivity (Dye Detection)

| Set | NG | NM | NL | NS | Neg. Urine | NTC |
|---|---|---|---|---|---|---|
| Set-1 | A | D | A | B | D | D |
| Set-6 | A | D | C | B | D | D |
| Set-10 | A | B | C | B | D | D |
| Set-11 | A | A | B | A | D | D |

Each of the primer sets showed cross reactivity with several of the closely related Neisseria species. To provide an additional level of specificity, molecular beacons targeting unique nucleotides within the N. gonorrhoeae amplicon were designed and utilized for detection (Table 5). Each molecular beacon probe was designed with 5′ fluorophore/3′ quencher modifications (6-Carboxyfluorescein (FAM) and Black Hole Quencher 1 (BHQ1)) included to provide target-specific fluorescent detection.

TABLE 5
Molecular Beacons

| ID | Fluor | Quench | Sequence (5′ to 3′) | Sequence ID |
|---|---|---|---|---|
| MB1 | FAM | BHQ1 | CAGGCCGGTTTTGCCGAAG | SEQ ID NO: 35 |
| | | | GACTGGTGTCGGCCTG | |
| MB2 | FAM | BHQ1 | CGCGAAGGACTGGTGTCGG | SEQ ID NO: 36 |
| | | | TACAGGACTTCGCG | |
| MB3 | FAM | BHQ1 | CGCGATCCACGAGCACTCT | SEQ ID NO: 69 |
| | | | TGCCAACACGATCGCG | |
| MB4 | FAM | BHQ1 | CGCGATCGGAGCACTCTTG | SEQ ID NO: 70 |
| | | | CCAACACGAAAGCGATCGC | |
| | | | G | |
| MB5 | FAM | BHQ1 | CGCGATCAGCACTCTGCCA | SEQ ID NO: 71 |
| | | | ACACGAAAGATCGCG | |
| MB6 | FAM | BHQ1 | CGCGATCCGTCCTGCGCGG | SEQ ID NO: 72 |
| | | | AAGATGTAACGGGATCGCG | |
| MB7 | FAM | BHQ1 | CTAGCGAAGGACTGGTGTC | SEQ ID NO: 92 |
| | | | GCTAG | |
| MBS | FAM | BHQ1 | CGC GAT GTT TGC CGA | SEQ ID NO: 93 |
| | | | AGG ACT GGT GTC ATC | |
| | | | GCG | |
| MB9 | FAM | BHQ1 | CGCGATCCTGCGCGGAAGA | SEQ ID NO: 94 |
| | | | TGTAACGATCGCG | |
| MB10 | FAM | BHQ1 | CGCGATCGCACGAGCACTC | SEQ ID NO: 95 |
| | | | TTGCCCGATCGCG | |
| MB11 | FAM | BHQ1 | CGCGATCACTTGTTTATTA | SEQ ID NO: 96 |
| | | | AAAACACGGATCGCG | |
| MB12 | FAM | BHQ1 | CGCGACCCGACTTGTTTAT | SEQ ID NO: 97 |
| | | | TAAAAACACGAGCACGGTC | |
| | | | GCG | |
| MB13 | FAM | BHQ1 | CGCGACCCCGACTTGTTTA | SEQ ID NO: 98 |
| | | | TTAAAAACACGAGCACGGG | |
| | | | TCGCG | |
| MB14 | FAM | BHQ1 | CGCGATCGAAGAAATTACA | SEQ ID NO: 99 |
| | | | ATTGATGGGCGTGATCGCG | |
| MB15 | FAM | BHQ1 | CGCGATCGAAGAAATTACA | SEQ ID NO: 100 |
| | | | ATTGATAGGCGGATCGCG | |
| MB16 | FAM | BHQ1 | CGCGATCAGCAGCCATCAT | SEQ ID NO: 101 |
| | | | TTAAAGAAAGGATCGCG | |

25 μl total volume reactions were using 10⁹ copies of gDNA of N. gonorrhoeae or closely related Neisseria species. Use of Molecular Beacons for detection resulted in a slight increase in reaction Tp, however the significant enhancement in assay specificity provided a reasonable tradeoff (Table 6). Results are classified by the time to positive (Tp) from reaction initiation as follows: “A” indicates a Tp of less than or equal to 9 minutes, “B” indicates a Tp of between 9 minutes and 15 minutes (inclusive), and “C” indicates a Tp of greater than 15 minutes or no amplification detected (No Call). An asterisk indicates an amplification curve with a shallow slope combined with a significantly reduced maximal fluorescence relative to N. gonorrhoeae reactions (i.e., no greater than 5%).

TABLE 6
Cross-Reactivity (Molecular Beacon)

| Primers | MB | Tp NG | Tp NM | Tp NL | Tp NS | Tp NTC |
|---|---|---|---|---|---|---|
| Set-1 | MB1 | A | C | C* | C | C* |
| Set-6 | MB2 | A | C | C* | C* | C* |
| Set-11 | MB3 | B | C | B | C | C |
| Set-11 | MB4 | B | C | B | C | C |
| Set-11 | MB5 | A | C | A | C | C |

### Example 4: Molecular Beacon Assay Kinetics

Typically, 3 to 5 μL of extracted nucleic acid material prepared as described (vide supra). 25 μl total volume reactions were prepared on ice as master mixes containing 1×NEB Isothermal amplification buffer supplemented with 5 mM KCl, 4.8 mM MgSO₄ and 1.6 mM each dCTP, dGTP, dATP and dTTP. NEB Bst2 polymerase (NEB CN #M0537L) and RTx Warmstart reverse transcriptase (NEB CN #M03805) were used at 8 and 7.5 units/reaction, respectively. Primers (2 μM inner primers, 0.2 μM outer primers, and 0.8 μM Loop primers) (Table 2) were added to individual reactions or directly to master mixes as required per experimental design along with one of the Molecular beacons (0.2 μM) (Table 5) was used for the detection of the amplified product. The reactions were incubated at 63° C. or 65° C. and kinetics were monitored using a Roche real-time Lightcycler96 (Roche). The time to positive for each primer-probe combination is reported in Table 7. Results are classified by the time to positive (Tp) from reaction initiation as follows: “A” indicates a Tp of less than or equal to 10 minutes, “B” indicates a Tp of between 10 minutes and 15 minutes (inclusive), and “C” indicates a Tp of greater than 15 minutes. “NT” indicates that this combination was not tested.

TABLE 7
Time to Positive Probe Detection

| Target | Primers | Beacon | 10³ IFU/mL | 10 IFU/mL |
|---|---|---|---|---|
| 23S | Set-8 | MB9 | A | B |
| 23S | Set-8 | MB6 | A | B |
| 23S | Set-9 | MB3 | B | C |
| 23S | Set-9 | MB10 | B | NT |
| 23S | Set-9 | MB4 | B | NT |
| 23S | Set-9 | MB5 | A | B |
| 23S | Set-10 | MB3 | C | C |
| 23S | Set-10 | MB10 | C | NT |
| 23S | Set-10 | MB4 | C | NT |
| rplF | Set-57 | MB14 | C^{†} | C^{‡} |
| rplF | Set-58 | MB14 | C | C |
| rplF | Set-59 | MB14 | B | B |
| rplF | Set-60 | MB14 | B | B |
| rplF | Set-61 | MB14 | C | C |
| rplF | Set-62 | MB15 | C | C |
| rsmB | Set-1 | MB1 | A | A |
| rsmB | Set-1 | MB7 | B | C |
| rsmB | Set-2 | MB7 | B | C |
| rsmB | Set-3 | MB1 | C | C |
| rsmB | Set-56 | MB1 | C | C |
| rsmB | Set-4 | MB1 | B | C |
| rsmB | Set-6 | MB1 | NT | A |
| rsmB | Set-6 | MB7 | NT | B |
| rsmB | Set-6 | MB2 | NT | B |
| rsmB | Set-6 | MB8 | NT | A |

^{†}100 CFU/ML;
^{‡}2 CFU/ml

Titred genomic DNA (gDNA, Zeptometrix, CN #0801482DNA-10UG) served as template for RTLAMP reactions. 25 μl total volume reactions were prepared on ice as master mixes containing 1×NEB Isothermal amplification buffer supplemented with 5 mM KCl, 4.8 mM MgSO₄ and 1.6 mM each dCTP, dGTP, dATP and dTTP. NEB Bst2 polymerase (NEB CN #M0537L) and RTx Warmstart reverse transcriptase (NEB CN #M0380S) were used at 8 and 7.5 units/reaction, respectively. Primers (2 μM inner primers, 0.2 M outer primers, and 0.8 μM Loop primers) (Table 2) were added to individual reactions or directly to master mixes as required per experimental design along with one of the Molecular beacons (0.2 μM) (Table 5) was used for the detection of the amplified product. The reactions were incubated at 63° C. or 65° C. and kinetics were monitored using a Roche real-time Lightcycler96 (Roche). The time to positive for each primer-probe combination is reported in Table 8. Results are classified by the time to positive (Tp) from reaction initiation as follows: “A” indicates a Tp of less than or equal to 10 minutes, “B” indicates a Tp of between 10 minutes and 15 minutes (inclusive), and “C” indicates a Tp of greater than 15 minutes.

TABLE 8
Time to Positive Probe Detection Genomic DNA

| Target | Primers | Beacon | Tp | amount gDNA |
|---|---|---|---|---|
| 23S | Set-8 | MB16 | C | 1.43 × 10⁵ |
| 23S | Set-9 | MB3 | B | 7 × 10⁶ |
| 23S | Set-9 | MB10 | B | 7 × 10⁶ |
| 23S | Set-9 | MB5 | A | 2 × 10⁵ |
| 23S | Set-10 | MB3 | C | 2 × 10⁵ |
| 23S | Set-10 | MB11 | C | 2 × 10⁵ |
| 23S | Set-10 | MB12 | C | 2 × 10⁵ |
| 23S | Set-10 | MB13 | C | 2 × 10⁵ |
| rsmB | Set-1 | MB1 | A | 6 × 10⁵ |
| rsmB | Set-2 | MB7 | C | 6 × 10⁵ |
| rsmB | Set-3 | MB1 | C | 6 × 10⁵ |
| rsmB | Set-56 | MB1 | C | 6 × 10⁵ |
| rsmB | Set-4 | MB1 | B | 6 × 10⁵ |
| rsmB | Set-6 | MB1 | A | 6 × 10⁵ |
| rsmB | Set-6 | MB7 | A | 6 × 10⁵ |
| rsmB | Set-6 | MB2 | A | 6 × 10⁵ |
| rsmB | Set-6 | MB8 | A | 6 × 10⁵ |

### Example 5: Assay Specificity

Potentially cross reacting organisms were tested and included common urinary tract and/or vaginal microbial colonizers and the closest N. gonorrhoeae phylogenetic relatives. Template input for amplification reactions was either from purified genomic DNA (gDNA) purchased from Zeptometrix at known concentrations or nucleic acids extracted from live bacterial or yeast cells. Except where indicated (*), live titred cells or known concentrations of genomic DNA were used as input for amplification reactions. In instances marked with an asterisk, where titred material and/or known concentrations were not available, template concentration was approximated based on RTqPCR standard curve Cq's. The assay was performed using Primer Set-1 and MB1 with RT-LAMP as described above. Positive calls were determined using the accompanying real time cycler standard analysis packages (Roche LightCycler 96 Software version 1.1.0.1320 or Bio-Rad CFX Manager Software version 3.1.1517.0823).

TABLE 9
Assay Specificity

| Organism | Nucleic acid source | Concentration | | % positive |
|---|---|---|---|---|
| Neisseria gonorrhoeae | extracted from cells | 1 × 10² | CFU × mL⁻¹ | 100 |
| Neisseria gonorrhoeae | purified gDNA | 1 × 10⁹ | copies × mL⁻¹ | 100 |
| Neisseria meningitidis | purified gDNA | 1 × 10⁹ | copies × mL⁻¹ | 0 |
| Neisseria lactamica | purified gDNA | 1 × 10⁹ | copies × mL⁻¹ | 0 |
| Neisseria sicca | purified gDNA | 1 × 10⁸ | copies × mL⁻¹ | 0 |
| Neisseria sicca | purified gDNA | 1 × 10⁹ | copies × mL⁻¹ | 50 |
| Neisseria sicca* | extracted from cells | 1 × 10⁷ | CFU × mL⁻¹ | 75 |
| Chlamydia trachomatis | extracted from cells | 1 × 10⁴ | CFU × mL⁻¹ | 0 |
| Escherichia coli* | extracted from cells | 1 × 10⁷ | CFU × mL⁻¹ | 0 |
| Proteus mirabilis* | extracted from cells | 1 × 10⁷ | CFU × mL⁻¹ | 0 |
| Candida albicans* | extracted from cells | 3.5 × 10⁵ | CFU × mL⁻¹ | 0 |
| Staphylococcus aureus* | extracted from cells | 5.3 × 10⁶ | CFU × mL⁻¹ | 0 |

| | | | |
|---|---|---|---|
| NTC | Nucleic acid free | Nuclease free H₂O | 0 |

For this assay, cross-reactive amplification was observed with N. sicca and N. lactamica nucleic acid material (Table 9). For N. sicca, amplification only occurred at concentrations above the FDA medically relevant recommendation of 1×10⁶ CFU×mL⁻¹ (U.S. Department of Health and Human Services, Food and Drug Administrations, 2011, Draft Guidance for Industry and Food and Drug Administration Staff; Establishing the Performance Characteristics of In Vitro Diagnostic Devices for Chlamydia trachomatis and/or Neisseria gonorrhoeae: Screening and Diagnostic Testing). In addition, even at the highest concentrations evaluated, N. sicca amplification was significantly delayed (≥16 minutes) relative to the average Tp for the same concentration of N. gonorrhoeae at times well beyond the assay cutoff. N. lactamica nucleic acid material amplification, in addition to a significant delay relative to N. gonorrhoeae, resulted in curves with a shallow slope and a significantly reduced maximal fluorescence relative to N. gonorrhoeae reactions. Using the associated Roche or Bio-Rad real-time cycler analysis packages (vide supra) for calling reactions as positive or negative, all other organisms tested resulted in negative calls.

### Example 6: Assay Sensitivity

Sensitivity of a variety of assays were also evaluated (Table 10, indicated CFU is per 50 μl extraction, 5 μl of which was used per reaction). Dilutions of titred N. gonorrhoeae stocks were prepared in PBS (1× diluted from 10×, Ambion CN #AM9624 in nuclease free water, Ambion, CN #AM9932) and spiked into neat urine samples followed by extraction using standard methods. Five μL of nucleic acid from the indicated total CFU per extraction served as template for assay RTLAMP reactions. As indicated in Table 10, most assays combined with Molecular Beacons for detection were sensitive to at least 5 CFU/extraction. Results are classified by the time to positive (Tp) from reaction initiation as follows: “A” indicates a Tp of less than or equal to 9 minutes, “B” indicates a Tp of between 9 minutes and 15 minutes (inclusive), “C” indicates a Tp of greater than 15 minutes or no amplification detected.

TABLE 10
Assay Sensitivity

| | Average Tp | | | | | |
|---|---|---|---|---|---|---|
| Assay | 0.5 CFU | 1 CFU | 5 CFU | 50 CFU | 500 CFU | NTC |
| Set-1, MB1 | B | A | A | A | A | C |
| Set-13, MB1 | NT | NT | C | NT | C | C |
| Set-4, MB1 | NT | NT | C | NT | C | C |
| Set-15, MB1 | NT | NT | C | NT | B | C |
| Set-6, MB2 | NT | NT | B | NT | NT | C |
| Set-17, MB6 | NT | A | A | A | A | C |
| Set-18, MB5 | B | B | B | B | B | C |

NT = Not Tested

For swab infused samples, an initial bench protocol was tested which included direct emersion of the swab into undiluted lysis buffer. Detection was very limited for the 1 CFU per extraction concentration (20%) and even more limited for the 0.5 CFU per extraction concentration (data not shown). The swab bench protocol was then adjusted to more closely mimic the urine extraction, specifically by including the same dilution of the lysis buffer with PBS as would result from addition of a urine specimen. This resulted in a 78% improvement, from 20% to 98%, in the frequency of 1 CFU extraction sample detection.

### Example 7: Limited Primer Sets

To assess the contribution of each primer set to the RTLAMP reaction, we also investigated use of just the inner primers or the inner primers plus the loop primers and compared those reactions to the complete 6 primer RTLAMP reaction, using a Molecular Beacon for detection. Table 11 provides an example using an assay comprised of Set-1 and MB1. Interestingly and noteworthy, the reaction still proceeds when the F3/B3 primers (set-19) are excluded. The absence of F3/B3 appears to have an impact on sensitivity, specifically consistency at low concentrations (Table 11, indicated CFU is per extraction, 5 uL of which was used per RTLAMP reaction). The reaction does not proceed if only the inner primers are included (Set-20). Results are classified by the time to positive (Tp) from reaction initiation as follows: “A” indicates a Tp of less than or equal to 9 minutes, “B” indicates a Tp of between 9 minutes and 15 minutes (inclusive), and “C” indicates a Tp of greater than 15 minutes or no amplification detected (No Call).

TABLE 11
Contribution of Primer Pairs

| | |
|---|---|
| Primer | Assay Tp |

| | | | | |
|---|---|---|---|---|
| Combination | 500 CFU | 5 CFU | 1 CFU | NTC |

| | | | | | |
|---|---|---|---|---|---|
| Set-1 | A | B | B | B | C |
| Set-19 | A | B | C | B | C |
| Set-20 | C | C | C | C | C |

Although the foregoing invention has been described in some detail by way of illustration and example for purposes of clarity of understanding, it is readily apparent to those of ordinary skill in the art in light of the teachings of this invention that certain changes and modifications can be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A composition comprising a set of polynucleotides selected from the group consisting of Set-1 through Set-55.

2. The composition of claim 1, further comprising a probe.

3.-13. (canceled)

14. A molecular beacon comprising a fluorophore, a quencher and a polynucleotide, wherein the polynucleotide is selected from the group consisting of: SEQ ID NO: 35, SEQ ID NO: 36. SEQ ID NO: 69 through SEQ ID NO: 72, and SEQ ID NO:
92 through SEQ ID NO: 101.

15.-16. (canceled)

17. A method of detecting Neisseria gonorrhoeae in a test sample, the method comprising:
a. extracting nucleic acid from the test sample;
b. amplifying a target sequence by reacting the nucleic acid extracted in step (a) with a reaction mixture comprising a strand displacement DNA polymerase and a sequence-specific primer set, wherein said sequence-specific primer set is selected from the group consisting of Set-1 through Set-55; and
c. detecting the presence or absence of an amplified product of step (b); wherein the presence of said amplification product is indicative of the presence of Neisseria gonorrhoeae in the test sample.

18. The method of claim 17, wherein the amplification in step (b) of the target sequence is performed at between about 60° C. and 67° C. for less than 30 minutes.

19. The method of claim 17, wherein the amplification step is performed for less than 15 minutes.

20. The method of claim 19, wherein the amplification step is performed for less than nine minutes.

21. The method of claim 17, wherein detecting the presence or absence of the amplification product comprises hybridizing the amplified product with a probe comprising a polynucleotide attached to a label.

22. The method of claim 21, wherein the polynucleotide comprises a sequence selected from the group consisting of SEQ ID NO: 72 (MB6), SEQ ID NO: 94 (MB9), and SEQ ID NO: 101 (MB16), and the sequence-specific primer set is selected from the group consisting of Set-8, Set-28, and Set-45.

23. The method of claim 21, wherein the polynucleotide comprises a sequence selected from the group consisting of SEQ ID NO: 69 (MB3), SEQ ID NO: 70 (MB4), SEQ ID NO: 71 (MB5), SEQ ID NO: 95 (MB10), SEQ ID NO: 96 (MB11), SEQ ID NO: 97 (MB12), and SEQ ID NO: 98 (MB13), and the sequence-specific primer set is selected from the group consisting of Set-9, Set-10, Set-11, Set-12, Set-29, Set-30, Set-31, Set-32, Set-46, Set-47, Set-48 and Set-49.

24. The method of claim 21, wherein the polynucleotide comprises a sequence selected from the group consisting of SEQ ID NO: 99 (MB14) and SEQ ID NO: 100 (MB15), and the sequence-specific primer set is selected from the group consisting of Set-57, Set-58, Set-59, Set-60, Set-61, Set-62, Set-64, Set-65, Set-66, Set-67, set-68, Set-69, Set-71, Set-72, Set-73, Set-74, Set-75, and Set-76.

25. The method of claim 21, wherein the polynucleotide comprises a sequence selected from the group consisting of SEQ ID NO: 35 (MB1), SEQ ID NO:36 (MB2), SEQ ID NO: 92 (MB7), and SEQ ID NO: 93 (MB8), and the sequence-specific primer set is selected from the group consisting of Set-1, Set-2, Set-3, Set-4, Set-5, Set-6, Set-19, Set-20, Set-21, Set-22, Set-23, Set-24, Set-25, Set-26, Set-39, Set-40, Set-41, Set-42, Set-43, Set-56, Set-63, and Set-70.

26. The method of claim 21, wherein the probe is a molecular beacon.

27. The method of claim 17, wherein the reaction mixture further comprises a reverse transcriptase.

28. The method of claim 17, wherein Neisseria gonorrhoeae is present in the test sample at a concentration of ≤100 CFU/mL.

29. The method of claim 28, wherein Neisseria gonorrhoeae is present in the test sample at a concentration of ≤10 CFU/mL.

30. A kit comprising the composition of claim 1 and amplification reagents.

31. The kit of claim 30, wherein the amplification reagents comprise a strand displacement polymerase.

32. The kit of claim 30, further comprising a probe.

33. A method of detecting Neisseria gonorrhoeae in a test sample, the method comprising:
a. extracting nucleic acid from the test sample;
b. amplifying a target sequence by reacting the nucleic acid extracted in step (a) for less than twenty minutes with a reaction mixture comprising a strand displacement DNA polymerase and a sequence-specific LAMP primer set; and
c. detecting the presence or absence of an amplified product of step (b); wherein the presence of said amplification product is indicative of the presence of Neisseria gonorrhoeae in the test sample.

34.-41. (canceled)


SEQUENCE LISTING (101 sequences)

SEQ ID NO: 1 - DNA, 18 bp, synthetic construct
  misc_feature 1..18: note = Description of Artificial Sequence: Synthetic primer
  source 1..18: mol_type = other DNA
atgccgaaagctatttgg

SEQ ID NO: 2 - DNA, 16 bp, synthetic construct
  misc_feature 1..16: note = Description of Artificial Sequence: Synthetic primer
  source 1..16: mol_type = other DNA
gccgtctttcgggtta

SEQ ID NO: 3 - DNA, 38 bp, synthetic construct
  misc_feature 1..38: note = Description of Artificial Sequence: Synthetic primer
  source 1..38: mol_type = other DNA
cttcttccaacgtaaccgcatagtggcggaaggtatcg

SEQ ID NO: 4 - DNA, 33 bp, synthetic construct
  misc_feature 1..33: note = Description of Artificial Sequence: Synthetic primer
  source 1..33: mol_type = other DNA
gccggtaaaccgtctgcccgaagtcctgtaccg

SEQ ID NO: 5 - DNA, 16 bp, synthetic construct
  misc_feature 1..16: note = Description of Artificial Sequence: Synthetic primer
  source 1..16: mol_type = other DNA
cgtccaacgccttagc

SEQ ID NO: 6 - DNA, 16 bp, synthetic construct
  misc_feature 1..16: note = Description of Artificial Sequence: Synthetic primer
  source 1..16: mol_type = other DNA
ttgccgaaggactggt

SEQ ID NO: 7 - DNA, 15 bp, synthetic construct
  misc_feature 1..15: note = Description of Artificial Sequence: Synthetic primer
  source 1..15: mol_type = other DNA
ctggtggcggaaggt

SEQ ID NO: 8 - DNA, 15 bp, synthetic construct
  misc_feature 1..15: note = Description of Artificial Sequence: Synthetic primer
  source 1..15: mol_type = other DNA
atccgttcgccgtct

SEQ ID NO: 9 - DNA, 31 bp, synthetic construct
  misc_feature 1..31: note = Description of Artificial Sequence: Synthetic primer
  source 1..31: mol_type = other DNA
ggcacggcttcttccaaatcgcggctaaggc

SEQ ID NO: 10 - DNA, 31 bp, synthetic construct
  misc_feature 1..31: note = Description of Artificial Sequence: Synthetic primer
  source 1..31: mol_type = other DNA
tttgccgaaggactggtgatacgccgcctgc

SEQ ID NO: 11 - DNA, 19 bp, synthetic construct
  misc_feature 1..19: note = Description of Artificial Sequence: Synthetic primer
  source 1..19: mol_type = other DNA
gtaaccgcatattcgtcca

SEQ ID NO: 12 - DNA, 17 bp, synthetic construct
  misc_feature 1..17: note = Description of Artificial Sequence: Synthetic primer
  source 1..17: mol_type = other DNA
tcggtacaggacttcgg

SEQ ID NO: 13 - DNA, 21 bp, synthetic construct
  misc_feature 1..21: note = Description of Artificial Sequence: Synthetic primer
  source 1..21: mol_type = other DNA
aagctatttggaaaaactggt

SEQ ID NO: 14 - DNA, 37 bp, synthetic construct
  misc_feature 1..37: note = Description of Artificial Sequence: Synthetic primer
  source 1..37: mol_type = other DNA
cttcttccaacgtaaccgcatagaaggtatcgcggct

SEQ ID NO: 15 - DNA, 30 bp, synthetic construct
  misc_feature 1..30: note = Description of Artificial Sequence: Synthetic primer
  source 1..30: mol_type = other DNA
cgtgccggtaaaccgtcgccgaagtcctgt

SEQ ID NO: 16 - DNA, 20 bp, synthetic construct
  misc_feature 1..20: note = Description of Artificial Sequence: Synthetic primer
  source 1..20: mol_type = other DNA
catattcgtccaacgcctta

SEQ ID NO: 17 - DNA, 15 bp, synthetic construct
  misc_feature 1..15: note = Description of Artificial Sequence: Synthetic primer
  source 1..15: mol_type = other DNA
tgccgaaggactggt

SEQ ID NO: 18 - DNA, 18 bp, synthetic construct
  misc_feature 1..18: note = Description of Artificial Sequence: Synthetic primer
  source 1..18: mol_type = other DNA
ttggacgaatatgcggtt

SEQ ID NO: 19 - DNA, 17 bp, synthetic construct
  misc_feature 1..17: note = Description of Artificial Sequence: Synthetic primer
  source 1..17: mol_type = other DNA
ccgtctgaaagcccaaa

SEQ ID NO: 20 - DNA, 34 bp, synthetic construct
  misc_feature 1..34: note = Description of Artificial Sequence: Synthetic primer
  source 1..34: mol_type = other DNA
agatacgccgcctgctgcgttggaagaagccgtg

SEQ ID NO: 21 - DNA, 35 bp, synthetic construct
  misc_feature 1..35: note = Description of Artificial Sequence: Synthetic primer
  source 1..35: mol_type = other DNA
tggaactggcggactgcggcgatattgtcctccac

SEQ ID NO: 22 - DNA, 18 bp, synthetic construct
  misc_feature 1..18: note = Description of Artificial Sequence: Synthetic primer
  source 1..18: mol_type = other DNA
ccgaagtcctgtaccgac

SEQ ID NO: 23 - DNA, 17 bp, synthetic construct
  misc_feature 1..17: note = Description of Artificial Sequence: Synthetic primer
  source 1..17: mol_type = other DNA
cgttaccgccttggaca

SEQ ID NO: 24 - DNA, 19 bp, synthetic construct
  misc_feature 1..19: note = Description of Artificial Sequence: Synthetic primer
  source 1..19: mol_type = other DNA
ttggacgaatatgcggtta

SEQ ID NO: 25 - DNA, 33 bp, synthetic construct
  misc_feature 1..33: note = Description of Artificial Sequence: Synthetic primer
  source 1..33: mol_type = other DNA
cgccgtctttcgggttaaggagccgtgccggta

SEQ ID NO: 26 - DNA, 41 bp, synthetic construct
  misc_feature 1..41: note = Description of Artificial Sequence: Synthetic primer
  source 1..41: mol_type = other DNA
gcatattttggaactggcggacgatattgtcctccacacgc

SEQ ID NO: 27 - DNA, 15 bp, synthetic construct
  misc_feature 1..15: note = Description of Artificial Sequence: Synthetic primer
  source 1..15: mol_type = other DNA
acgccgaagtcctgt

SEQ ID NO: 28 - DNA, 16 bp, synthetic construct
  misc_feature 1..16: note = Description of Artificial Sequence: Synthetic primer
  source 1..16: mol_type = other DNA
cgttaccgccttggac

SEQ ID NO: 29 - DNA, 21 bp, synthetic construct
  misc_feature 1..21: note = Description of Artificial Sequence: Synthetic primer
  source 1..21: mol_type = other DNA
gcaatgccgaaagctatttgg

SEQ ID NO: 30 - DNA, 20 bp, synthetic construct
  misc_feature 1..20: note = Description of Artificial Sequence: Synthetic primer
  source 1..20: mol_type = other DNA
aaatccgttcgccgtctttc

SEQ ID NO: 31 - DNA, 41 bp, synthetic construct
  misc_feature 1..41: note = Description of Artificial Sequence: Synthetic primer
  source 1..41: mol_type = other DNA
ggcttcttccaacgtaaccgcattggtggcggaaggtatcg

SEQ ID NO: 32 - DNA, 38 bp, synthetic construct
  misc_feature 1..38: note = Description of Artificial Sequence: Synthetic primer
  source 1..38: mol_type = other DNA
tgcccggttttgccgaaggtaaggagatacgccgcctg

SEQ ID NO: 33 - DNA, 20 bp, synthetic construct
  misc_feature 1..20: note = Description of Artificial Sequence: Synthetic primer
  source 1..20: mol_type = other DNA
attcgtccaacgccttagcc

SEQ ID NO: 34 - DNA, 21 bp, synthetic construct
  misc_feature 1..21: note = Description of Artificial Sequence: Synthetic primer
  source 1..21: mol_type = other DNA
actggtgtcggtacaggactt

SEQ ID NO: 35 - DNA, 35 bp, synthetic construct
  misc_feature 1..35: note = Description of Artificial Sequence: Synthetic probe
  source 1..35: mol_type = other DNA
caggccggttttgccgaaggactggtgtcggcctg

SEQ ID NO: 36 - DNA, 33 bp, synthetic construct
  misc_feature 1..33: note = Description of Artificial Sequence: Synthetic probe
  source 1..33: mol_type = other DNA
cgcgaaggactggtgtcggtacaggacttcgcg

SEQ ID NO: 37 - DNA, 18 bp, synthetic construct
  misc_feature 1..18: note = Description of Artificial Sequence: Synthetic primer
  source 1..18: mol_type = other DNA
gagcgcaacccttgtcat

SEQ ID NO: 38 - DNA, 19 bp, synthetic construct
  misc_feature 1..19: note = Description of Artificial Sequence: Synthetic primer
  source 1..19: mol_type = other DNA
tccgacttcatgcactcga

SEQ ID NO: 39 - DNA, 42 bp, synthetic construct
  misc_feature 1..42: note = Description of Artificial Sequence: Synthetic primer
  source 1..42: mol_type = other DNA
gaggacttgacgtcatccccacgggcactctaatgagactgc

SEQ ID NO: 40 - DNA, 42 bp, synthetic construct
  misc_feature 1..42: note = Description of Artificial Sequence: Synthetic primer
  source 1..42: mol_type = other DNA
atggtcggtacagagggtagccagtgcaatccggactacgat

SEQ ID NO: 41 - DNA, 20 bp, synthetic construct
  misc_feature 1..20: note = Description of Artificial Sequence: Synthetic primer
  source 1..20: mol_type = other DNA
cttcctccggcttgtcaccg

SEQ ID NO: 42 - DNA, 23 bp, synthetic construct
  misc_feature 1..23: note = Description of Artificial Sequence: Synthetic primer
  source 1..23: mol_type = other DNA
aggcggagccaatctcacaaaac

SEQ ID NO: 43 - DNA, 18 bp, synthetic construct
  misc_feature 1..18: note = Description of Artificial Sequence: Synthetic primer
  source 1..18: mol_type = other DNA
ccggctaaggtcccaaat

SEQ ID NO: 44 - DNA, 19 bp, synthetic construct
  misc_feature 1..19: note = Description of Artificial Sequence: Synthetic primer
  source 1..19: mol_type = other DNA
tcgcacttctgatacctcc

SEQ ID NO: 45 - DNA, 40 bp, synthetic construct
  misc_feature 1..40: note = Description of Artificial Sequence: Synthetic primer
  source 1..40: mol_type = other DNA
tcgaccagtgagctattacgctgaagtgggaaggcacaga

SEQ ID NO: 46 - DNA, 41 bp, synthetic construct
  misc_feature 1..41: note = Description of Artificial Sequence: Synthetic primer
  source 1..41: mol_type = other DNA
ctataaccgaagctgcggatgcatcagcctacagaacgctc

SEQ ID NO: 47 - DNA, 21 bp, synthetic construct
  misc_feature 1..21: note = Description of Artificial Sequence: Synthetic primer
  source 1..21: mol_type = other DNA
gcttctaagccaacatcctgg

SEQ ID NO: 48 - DNA, 19 bp, synthetic construct
  misc_feature 1..19: note = Description of Artificial Sequence: Synthetic primer
  source 1..19: mol_type = other DNA
cggtttaccggcatggtag

SEQ ID NO: 49 - DNA, 20 bp, synthetic construct
  misc_feature 1..20: note = Description of Artificial Sequence: Synthetic primer
  source 1..20: mol_type = other DNA
agagaactcgggagaaggaa

SEQ ID NO: 50 - DNA, 20 bp, synthetic construct
  misc_feature 1..20: note = Description of Artificial Sequence: Synthetic primer
  source 1..20: mol_type = other DNA
tcgctaccttaggaccgtta

SEQ ID NO: 51 - DNA, 43 bp, synthetic construct
  misc_feature 1..43: note = Description of Artificial Sequence: Synthetic primer
  source 1..43: mol_type = other DNA
cagccacctattctctgcgaccggagaaggtatgccctctaag

SEQ ID NO: 52 - DNA, 40 bp, synthetic construct
  misc_feature 1..40: note = Description of Artificial Sequence: Synthetic primer
  source 1..40: mol_type = other DNA
cgtatagggtgtaacgcctgccggcttcgatccgatgctt

SEQ ID NO: 53 - DNA, 19 bp, synthetic construct
  misc_feature 1..19: note = Description of Artificial Sequence: Synthetic primer
  source 1..19: mol_type = other DNA
ggcttacggagcaagtcct

SEQ ID NO: 54 - DNA, 20 bp, synthetic construct
  misc_feature 1..20: note = Description of Artificial Sequence: Synthetic primer
  source 1..20: mol_type = other DNA
cggtgccggaaggttaattg

SEQ ID NO: 55 - DNA, 18 bp, synthetic construct
  misc_feature 1..18: note = Description of Artificial Sequence: Synthetic primer
  source 1..18: mol_type = other DNA
ggagaaggaactcggcaa

SEQ ID NO: 56 - DNA, 18 bp, synthetic construct
  misc_feature 1..18: note = Description of Artificial Sequence: Synthetic primer
  source 1..18: mol_type = other DNA
cttcgatccgatgcttgc

SEQ ID NO: 57 - DNA, 41 bp, synthetic construct
  misc_feature 1..41: note = Description of Artificial Sequence: Synthetic primer
  source 1..41: mol_type = other DNA
agccacctattctctgcgaccggagaaggtatgccctctaa

SEQ ID NO: 58 - DNA, 40 bp, synthetic construct
  misc_feature 1..40: note = Description of Artificial Sequence: Synthetic primer
  source 1..40: mol_type = other DNA
gagcactcttgccaacacgaacaattaaccttccggcacc

SEQ ID NO: 59 - DNA, 22 bp, synthetic construct
  misc_feature 1..22: note = Description of Artificial Sequence: Synthetic primer
  source 1..22: mol_type = other DNA
cttacggagcaagtccttaacc

SEQ ID NO: 60 - DNA, 20 bp, synthetic construct
  misc_feature 1..20: note = Description of Artificial Sequence: Synthetic primer
  source 1..20: mol_type = other DNA
gtatagggtgtaacgcctgc

SEQ ID NO: 61 - DNA, 38 bp, synthetic construct
  misc_feature 1..38: note = Description of Artificial Sequence: Synthetic primer
  source 1..38: mol_type = other DNA
agcactctgccaacacgaaccacggccttccaattaac

SEQ ID NO: 62 - DNA, 20 bp, synthetic construct
  misc_feature 1..20: note = Description of Artificial Sequence: Synthetic primer
  source 1..20: mol_type = other DNA
gtatagggtgtcacgcctgc

SEQ ID NO: 63 - DNA, 19 bp, synthetic construct
  misc_feature 1..19: note = Description of Artificial Sequence: Synthetic primer
  source 1..19: mol_type = other DNA
agagaataggtggctgcga

SEQ ID NO: 64 - DNA, 18 bp, synthetic construct
  misc_feature 1..18: note = Description of Artificial Sequence: Synthetic primer
  source 1..18: mol_type = other DNA
agacagtgtggccatcgt

SEQ ID NO: 65 - DNA, 47 bp, synthetic construct
  misc_feature 1..47: note = Description of Artificial Sequence: Synthetic primer
  source 1..47: mol_type = other DNA
gcaggcgtcacaccctatacggtttattaaaaacacagcactctgcc

SEQ ID NO: 66 - DNA, 37 bp, synthetic construct
  misc_feature 1..37: note = Description of Artificial Sequence: Synthetic primer
  source 1..37: mol_type = other DNA
cggtaaacggcggccgtaaattcgtgcgggtcggaac

SEQ ID NO: 67 - DNA, 22 bp, synthetic construct
  misc_feature 1..22: note = Description of Artificial Sequence: Synthetic primer
  source 1..22: mol_type = other DNA
ctatacgtccactttcgtgttg

SEQ ID NO: 68 - DNA, 21 bp, synthetic construct
  misc_feature 1..21: note = Description of Artificial Sequence: Synthetic primer
  source 1..21: mol_type = other DNA
taacggtcctaaggtagcgaa

SEQ ID NO: 69 - DNA, 35 bp, synthetic construct
  misc_feature 1..35: note = Description of Artificial Sequence: Synthetic probe
  source 1..35: mol_type = other DNA
cgcgatccacgagcactcttgccaacacgatcgcg

SEQ ID NO: 70 - DNA, 39 bp, synthetic construct
  misc_feature 1..39: note = Description of Artificial Sequence: Synthetic probe
  source 1..39: mol_type = other DNA
cgcgatcggagcactcttgccaacacgaaagcgatcgcg

SEQ ID NO: 71 - DNA, 34 bp, synthetic construct
  misc_feature 1..34: note = Description of Artificial Sequence: Synthetic probe
  source 1..34: mol_type = other DNA
cgcgatcagcactctgccaacacgaaagatcgcg

SEQ ID NO: 72 - DNA, 38 bp, synthetic construct
  misc_feature 1..38: note = Description of Artificial Sequence: Synthetic probe
  source 1..38: mol_type = other DNA
cgcgatccgtcctgcgcggaagatgtaacgggatcgcg

SEQ ID NO: 73 - DNA, 27 bp, synthetic construct
  misc_feature 1..27: note = Description of Artificial Sequence: Synthetic primer
  source 1..27: mol_type = other DNA
ccgtctgcccggtcgccgaagtcctgt

SEQ ID NO: 74 - DNA, 19 bp, synthetic construct
  misc_feature 1..19: note = Description of Artificial Sequence: Synthetic primer
  source 1..19: mol_type = other DNA
ttggaacagaggcattggt

SEQ ID NO: 75 - DNA, 21 bp, synthetic construct
  misc_feature 1..21: note = Description of Artificial Sequence: Synthetic primer
  source 1..21: mol_type = other DNA
caacttgtttatccgagccag

SEQ ID NO: 76 - DNA, 42 bp, synthetic construct
  misc_feature 1..42: note = Description of Artificial Sequence: Synthetic primer
  source 1..42: mol_type = other DNA
gctgactaatgcgcgagcaggcattctgatgtagccattgaa

SEQ ID NO: 77 - DNA, 44 bp, synthetic construct
  misc_feature 1..44: note = Description of Artificial Sequence: Synthetic primer
  source 1..44: mol_type = other DNA
ggttatcgtgctcaagcacaaggtctgtttggctaggagtttga

SEQ ID NO: 78 - DNA, 22 bp, synthetic construct
  misc_feature 1..22: note = Description of Artificial Sequence: Synthetic primer
  source 1..22: mol_type = other DNA
accagacattgcatttgcttgt

SEQ ID NO: 79 - DNA, 18 bp, synthetic construct
  misc_feature 1..18: note = Description of Artificial Sequence: Synthetic primer
  source 1..18: mol_type = other DNA
tgcctgaaggtgtctccg

SEQ ID NO: 80 - DNA, 20 bp, synthetic construct
  misc_feature 1..20: note = Description of Artificial Sequence: Synthetic primer
  source 1..20: mol_type = other DNA
gcattctgatgtagccattg

SEQ ID NO: 81 - DNA, 18 bp, synthetic construct
  misc_feature 1..18: note = Description of Artificial Sequence: Synthetic primer
  source 1..18: mol_type = other DNA
ctctgtttggctaggagt

SEQ ID NO: 82 - DNA, 44 bp, synthetic construct
  misc_feature 1..44: note = Description of Artificial Sequence: Synthetic primer
  source 1..44: mol_type = other DNA
tcttctcaaaaccttctgaaacaccaagcaaatgcaatgtctgg

SEQ ID NO: 83 - DNA, 38 bp, synthetic construct
  misc_feature 1..38: note = Description of Artificial Sequence: Synthetic primer
  source 1..38: mol_type = other DNA
cgtgctcaagcacaaggtaattgaacggagacaccttc

SEQ ID NO: 84 - DNA, 21 bp, synthetic construct
  misc_feature 1..21: note = Description of Artificial Sequence: Synthetic primer
  source 1..21: mol_type = other DNA
accatattgctgactaatgcg

SEQ ID NO: 85 - DNA, 22 bp, synthetic construct
  misc_feature 1..22: note = Description of Artificial Sequence: Synthetic primer
  source 1..22: mol_type = other DNA
atccgatcgtatatgaaatgcc

SEQ ID NO: 86 - DNA, 19 bp, synthetic construct
  misc_feature 1..19: note = Description of Artificial Sequence: Synthetic primer
  source 1..19: mol_type = other DNA
gcagtaaacaagcaaatgc

SEQ ID NO: 87 - DNA, 43 bp, synthetic construct
  misc_feature 1..43: note = Description of Artificial Sequence: Synthetic primer
  source 1..43: mol_type = other DNA
tcttctcaaaaccttctgaaacaccaatgtctggtactgctcg

SEQ ID NO: 88 - DNA, 43 bp, synthetic construct
  misc_feature 1..43: note = Description of Artificial Sequence: Synthetic primer
  source 1..43: mol_type = other DNA
cttctcaaaaccttctgaaacacctaatgtctggtactgctcg

SEQ ID NO: 89 - DNA, 44 bp, synthetic construct
  misc_feature 1..44: note = Description of Artificial Sequence: Synthetic primer
  source 1..44: mol_type = other DNA
tcttctcaaaaccttctgaaacaccgcagtaaacaagcaaatgc

SEQ ID NO: 90 - DNA, 27 bp, synthetic construct
  misc_feature 1..27: note = Description of Artificial Sequence: Synthetic primer
  source 1..27: mol_type = other DNA
ccattgaatttaatgatggcaaattga

SEQ ID NO: 91 - DNA, 39 bp, synthetic construct
  misc_feature 1..39: note = Description of Artificial Sequence: Synthetic primer
  source 1..39: mol_type = other DNA
cgtgggttatcgtgctcaagcttgaacggagacaccttc

SEQ ID NO: 92 - DNA, 24 bp, synthetic construct
  misc_feature 1..24: note = Description of Artificial Sequence: Synthetic probe
  source 1..24: mol_type = other DNA
ctagcgaaggactggtgtcgctag

SEQ ID NO: 93 - DNA, 33 bp, synthetic construct
  misc_feature 1..33: note = Description of Artificial Sequence: Synthetic probe
  source 1..33: mol_type = other DNA
cgcgatgtttgccgaaggactggtgtcatcgcg

SEQ ID NO: 94 - DNA, 32 bp, synthetic construct
  misc_feature 1..32: note = Description of Artificial Sequence: Synthetic probe
  source 1..32: mol_type = other DNA
cgcgatcctgcgcggaagatgtaacgatcgcg

SEQ ID NO: 95 - DNA, 32 bp, synthetic construct
  misc_feature 1..32: note = Description of Artificial Sequence: Synthetic probe
  source 1..32: mol_type = other DNA
cgcgatcgcacgagcactcttgcccgatcgcg

SEQ ID NO: 96 - DNA, 34 bp, synthetic construct
  misc_feature 1..34: note = Description of Artificial Sequence: Synthetic probe
  source 1..34: mol_type = other DNA
cgcgatcacttgtttattaaaaacacggatcgcg

SEQ ID NO: 97 - DNA, 41 bp, synthetic construct
  misc_feature 1..41: note = Description of Artificial Sequence: Synthetic probe
  source 1..41: mol_type = other DNA
cgcgacccgacttgtttattaaaaacacgagcacggtcgcg

SEQ ID NO: 98 - DNA, 43 bp, synthetic construct
  misc_feature 1..43: note = Description of Artificial Sequence: Synthetic probe
  source 1..43: mol_type = other DNA
cgcgaccccgacttgtttattaaaaacacgagcacgggtcgcg

SEQ ID NO: 99 - DNA, 38 bp, synthetic construct
  misc_feature 1..38: note = Description of Artificial Sequence: Synthetic probe
  source 1..38: mol_type = other DNA
cgcgatcgaagaaattacaattgatgggcgtgatcgcg

SEQ ID NO: 100 - DNA, 37 bp, synthetic construct
  misc_feature 1..37: note = Description of Artificial Sequence: Synthetic probe
  source 1..37: mol_type = other DNA
cgcgatcgaagaaattacaattgataggcggatcgcg

SEQ ID NO: 101 - DNA, 36 bp, synthetic construct
  misc_feature 1..36: note = Description of Artificial Sequence: Synthetic probe
  source 1..36: mol_type = other DNA
cgcgatcagcagccatcatttaaagaaaggatcgcg